(19) United States
(12) Patent Application Publication
(10) Pub. No.: US 2004/0147803 A1
(43) Pub. Date: Jul. 29, 2004

(54) Vascular assist device and methods

(76) Inventors: Anant V. Hegde, Newark, CA (US); Wally S. Buch, Atherton, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/681,821

(22) Filed: Oct. 7, 2003

Related U.S. Application Data
(60) Provisional application No. 60/416,477, filed on Oct. 7, 2002.

(51) Int. Cl. A61M 1/10

(52) U.S. Cl. 600/16

(57) ABSTRACT

A vascular assist device is provided that can be readily implanted within the body of the patient without involving direct blood contact. The device is readily repositioned and/or removed. A method of fabrication and a method of implanting such a device is provided. A method of use is provided including increasing the pressure in an aortic cuff based on a control signal related to systole and/or diastole of a heart and/or the aortic pressure.

### CROSS-REFERENCE TO RELATED APPLICATIONS

This application claims priority under 35 U.S.C. 119 to provisional application serial No. 60/416,477, filed on Oct. 7, 2002, and entitled “Vascular Assist Device” the entirety of which is incorporated herein by reference.

### BACKGROUND

1. Field of the Invention

The present invention is directed to vascular assist devices and methods, and more particularly directed to vascular assist devices that are readily fabricated, installed, adjusted and removed.

2. Description of the Related Art

Congestive heart failure is a condition that causes the heart to pump less efficiently. Typically the heart has been weakened over time by an underlying problem, such as clogged arteries, high blood pressure, a defect in its muscular walls or valves, or some other medical condition. The body depends on the heart's pumping action to deliver oxygen and nutrient-rich blood so it can function normally. In people with congestive heart failure, the body fails to get an adequate supply. As a result, they tend to feel weak, fatigued, or short of breath. Everyday activities such as walking, climbing stairs, carrying groceries and yard work can become quite difficult.

Congestive heart failure develops over time. The slow onset and progression of congestive heart failure is caused by the heart's own efforts to compensate for the weakening of the heart muscles. The heart tries to compensate for the weakening by enlarging and forcing a faster pumping rate to move more blood through the vasculature of the body.

If the left side of the heart is not working properly, blood and other fluids back up into the lungs leading to the shortness of breath and exhaustion discussed above. If the right side of the heart is not working properly, the slow blood flow causes build up of fluid in the veins causing the legs and ankles to show signs of swelling (edema). Edema often spreads to the lungs, liver, and stomach. Such a fluid buildup may also cause kidney failure due to the body's ability to dispose of salt and water. As heart failure progresses, a patient's heart becomes weaker and the symptoms begin to manifest.

People at risk for congestive heart failure may undertake various therapies to ease the workload of the heart. Such treatment may include lifestyle changes, medicines, transcatheter interventions, and surgery. While lifestyle changes and medicines are often effective non-invasive procedures that can be undertaken, they are not as effective as the alternative, albeit more invasive, procedures. That being said, transcatheter interventions and surgical procedures are highly invasive and can create substantial risk in more delicate patients (e.g., elderly people, obese people, etc.).

Examples of transcatheter interventions include angioplasty, stenting, and inotropic drug therapy. Surgical procedures include heart valve repair or replacement, pacemaker insertion, correction of congenital heart defects, coronary artery bypass surgery, mechanical assist devices, and heart transplant.

When the heart can no longer adequately function and a patient is at risk of dying it is referred to as end-stage congestive heart failure. In such cases heart transplants are often required. Mechanical assist devices such as ventricular assist devices (VADs) and axial pumps have proven to be effective in offloading the workload of the heart. These devices can act as a temporary assist for a patient's heart prior to transplant. Studies have shown that approximately twenty percent (20%) of people using VADs have recovered or healed by offloading the heart for some period of time.

Recently, ventricular assist devices have been considered as an alternative to heart transplant and have been successfully implanted in several patients worldwide. Ventricular assist devices are able to totally offload the heart, potentially leading to recovery of the heart.

There are several types of ventricular assist devices. Left ventricular assist devices that offload the left ventricle of the heart, right ventricular assist devices that offload the right ventricle of the heart and atrial assist devices that offload the atrium of the heart. These devices come into direct contact with the blood. Such direct blood contact is a major concern with respect to thrombus formation and it is necessary to give blood thinners and anticoagulants to patients fitted with such ventricular assist devices. To insert such a device it is necessary to make incisions in the heart chambers and aorta, thereby leading to infection at the implant site as well as around the conduits connecting to external devices.

Another type of assist device is the intra-aortic balloon pump (IABP). IABPs provide assistance by decreasing myocardial oxygen consumption by reducing heart afterload, as well as increasing coronary artery profusion by augmenting diastolic coronary artery flow. IABPs do not require surgical intervention to install, but rather is placed through an open approach to the common femoral artery.

Another device that is often used is an impeller, which is a miniature pump catheter that continuously pumps the blood. Aortomyplasty is another way to augment the diastolic pressure and increase coronary artery flow.

To avoid the problems of biomaterial interface and to avoid disadvantages of other known methods of increasing blood flow, devices that compress the aorta externally were developed. Such devices may often include rigid mechanical jaws that are not compliant, thereby increasing the likelihood of injury to the aorta. Additionally such devices limit the mobility of patients, thus compromising the quality of life.

Conventional vascular assist devices are often configured to increase arterial blood flow from the heart. Generally speaking, many conventional vascular assist devices are both difficult to install and cumbersome for the patient. Several vascular assist devices are configured to be inserted into the vasculature, thereby causing potential infection and other related difficulties. Other devices that are configured to be installed externally to the vasculature include many components that need to be installed in very small areas. Moreover, when the devices need to be adjusted and/or removed, complex procedures are required. Moreover, such devices also are not synchronized with the cardiac cycle, thereby not appropriately timing the compression of the aorta.

### SUMMARY OF THE INVENTION

In light of the previously described problems associated with conventional vascular assist devices, one object of the embodiments of the present invention is to provide a vascular assist device that can be readily implanted within the body of the patient without involving direct blood contact. The device is also readily repositioned and/or removed.

In one embodiment, there is provided a device including a compliant first layer configured to engage internal vasculature and a second layer coupled to the first layer. The second layer has a stiffness greater than a stiffness of the first layer. The first layer and the second layer define a cavity between them. The cavity has a volume and the first layer is configured to be deformed in response to a change in the volume of the cavity.

In another embodiment, there is provided a device including an expandable layer configured to engage internal vasculature; and a cover layer coupled to the expandable layer. The cover layer has a length and a width. The expandable layer and the cover layer together define a cavity having a volume. The cover layer defines an opening in fluid communication with the cavity. A reinforcement element is coupled to the cover layer and configured to maintain the length and width of the cover layer. The expandable layer is configured to be selectively deformed in response to a change in the volume of the cavity.

In another embodiment there is provided a vascular assist device configured to be coupled to at least a portion of a blood vessel including a vascular engaging layer; an expandable layer; and a cover layer. The device has an uninstalled configuration and an installed configuration. The vascular engaging layer is positioned between the outer wall of the blood vessel and the expanding layer. The cover layer and the expanding layer are coupled so as to form a cavity therebetween. The cavity is bounded by the expanding layer and the cover layer. The cover layer has an opening that is in communication with the cavity. The cavity is configured to selectively receive a fluid via the opening. The fluid causes the volume of the cavity to change such that the change in cavity volume causes the expanding layer to deform more than the cover layer to accommodate the change in cavity volume.

In another embodiment, there is provided a vascular assist device configured to be coupled to at least a portion of a blood vessel. The vascular assist device includes a vascular engaging layer having a first stiffness; a cover layer having a second stiffness that is greater than the first stiffness and being coupled to the vascular engaging layer such that a portion of the cover layer extends past at least a portion of the perimeter of the vascular engaging layer. The cover layer and the vascular engaging layer form a cavity. The cover layer has an opening that is in communication with the cavity. The cavity is configured to selectively receive a fluid via the opening. The device has an uninstalled configuration and an installed configuration.

In another embodiment there is provided a system that includes a pump having a controller configured to receive a signal associated with the cardiac cycle of a heart and a cuff. The cuff includes a compliant first layer configured to engage internal vasculature; a second layer coupled to the first layer and having a stiffness greater than a stiffness of the first layer and having an opening formed therein. The compliant first layer and the second layer being coupled to form a cavity bounded by the first layer and the second layer. The cavity being in communication with the opening in the second layer. A conduit coupled between the opening and the pump. The conduit is configured to convey a fluid between the pump and the cavity thereby causing deformation of the first layer by expanding and contracting the cavity.

In another embodiment there is provided a method for augmenting blood flow in a patient body using a cuff formed from a first layer joined to a second layer so that a cavity exists between the layers such that filling the cavity preferentially deforms the first layer. The method includes detecting a first cardiac cycle trigger; porting a fluid into the cavity so as to elastically deform the first layer thereby compressing a blood vessel in response to the first cardiac cycle trigger; and porting a fluid out of the cavity in response to a second cardiac cycle trigger.

In another embodiment there is provided a method for augmenting blood flow in a body using a cuff formed from a first layer joined to a second layer so that a cavity exists between the layers such that filling the cavity preferentially deforms the first layer. The method includes detecting a cardiac cycle trigger; porting a fluid into the cavity so as to elastically deform the first layer thereby compressing a blood vessel in response to the cardiac cycle trigger; holding the vessel compressed for known duration and porting a fluid out of the cavity at the end of the duration in order to allow the vessel to relax.

In another embodiment there is provided a method for augmenting blood flow in a vessel of a patient using a cuff having a compliant first layer that at least partially encircles a vessel adjacent the cuff, a second layer coupled to the first layer, the first layer and the second layer defining a cavity. The method includes changing the pressure of a fluid in the cavity based on a signal associated with the cardiac cycle; deforming the first layer in response to the changing pressure of the fluid in the cavity; and deforming the walls of a vessel at least partially encircled by the first layer in response to the deforming of the first layer.

In another embodiment there is provided a system including a plurality of cuffs, each of the plurality of cuffs including a compliant first layer configured to engage internal vasculature; a second layer coupled to the first layer, the first layer and the second layer defining a cavity therebetween, the second layer defining an opening in communication with the cavity; and a connector configured to couple the plurality of cuffs to one another.

Another object of the embodiments of the present invention is to provide a method of fabrication and a method of implanting such a vascular assist device.

A further object of the embodiments of the present invention is to provide a method including increasing a pressure of a liquid or gas in an aortic cuff based on a control signal related to the systole and/or diastole of the heart and/or the aortic pressure.

Other objects, advantages and features associated with the embodiments of the present invention will become more readily apparent to those skilled in the art from the following detailed description. As will be realized, the invention is capable of other and different embodiments and its several details are capable of modification in various obvious aspects, all without departing from the invention. Accordingly, the drawings and the description are regarded as illustrative in nature, and not limiting.

### BRIEF DESCRIPTION OF THE DRAWINGS

The present invention is described with reference to the accompanying drawings. In the drawings, like reference numbers indicate similar elements.

FIG. 1 is a schematic diagram of the ventricular assist device system in accordance with one embodiment of the present invention.

FIG. 2 is an exploded perspective view of the ventricular assist device in accordance with an embodiment of the present invention.

FIG. 3 is a perspective view of the device illustrated in FIG. 2 in a first configuration.

FIG. 4 is a cross-sectional view of the device illustrated in FIG. 3 taken along the line of 5-5 in FIG. 3.

FIG. 4A is a cross-sectional view of an alternative embodiment of the device illustrated in FIG. 3.

FIGS. 5A and 5B are perspective views of alternative configurations of embodiments of the cover layer and expandable layer.

FIGS. 6A, 6B and 6C are views of an embodiment of the expandable layer.

FIG. 7 is a perspective view of a device in accordance with an embodiment of the present invention positioned on the ascending aorta, the device being in a second configuration.

FIG. 8A is a perspective view of a device in accordance with an embodiment of the present invention positioned in an uninstalled configuration.

FIG. 8B is a perspective view of the device in FIG. 8A where an embodiment of a material layer is exposed.

FIG. 9 illustrates another embodiment of the device of FIG. 8A according to the present invention in a second configuration around the ascending aorta.

FIG. 10 is a perspective view of an device according to an embodiment of the present invention having an embodiment of a vascular engaging layer.

FIG. 11is a cross section view of the device of FIG. 10.

FIG. 12 illustrates the device of FIG. 10 in an installed configuration about the ascending aorta.

FIG. 13 illustrates a device of the present invention having an alternative embodiment of the vascular engaging layer in an installed configuration about the ascending aorta.

FIG. 14A illustrates an embodiment of a segmented cuff according to the present invention in an uninstalled configuration.

FIG. 14B illustrates a section of the segmented cuff embodiment of FIG. 14A.

FIGS. 15A and 15B illustrate segmented cuff embodiments of the present invention having embodiments of alternative tab profiles of the present invention.

FIGS. 16A-16D illustrate perspective and cross section views of alternative segmented cuff embodiments of the present invention.

FIG. 17-FIG. 18B illustrate perspective views of alternative segmented cuff embodiments of the present invention.

FIG. 18C illustrates a cross section view of an embodiment of a conduit of the present invention having a variable diameter.

FIGS. 19A and 19B illustrate section views of an embodiment of a fluid volume compensator of the present invention.

FIGS. 19C and 19D illustrate perspective views of alternative embodiments of the vascular assist ssytem of the present invention.

FIGS. 20A-29 illustrate various alternative embodiments of connection mechanisms for the various devices according to the present invention.

FIG. 30 illustrates an alternative configuration of a device according to the present invention having an alternative embodiment of a fastening system of the present invention.

FIG. 31 illustrates representative pressure and ECG waves generated by an embodiment of the vascular assist sysstem of the present invention operated in coplusation mode.

FIG. 32 illustrates representative pressure and ECG waves generated by an embodiment of the vascular assist sysstem of the present invention operated in counterplusation mode.

### DETAILED DESCRIPTION

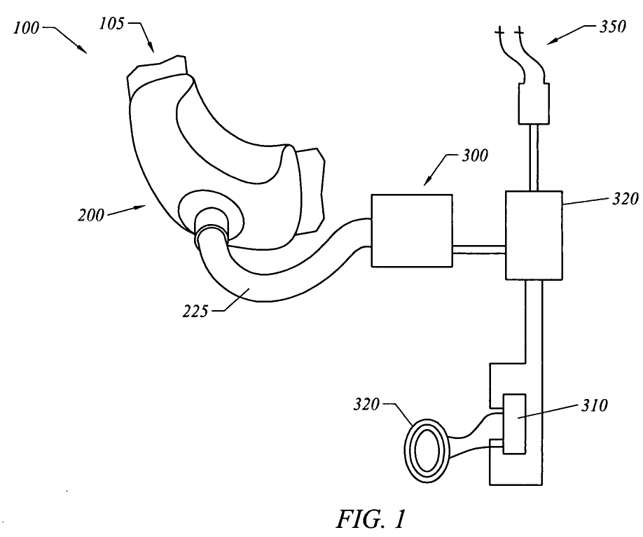

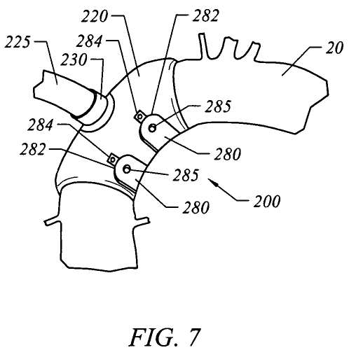

FIG. 1 illustrates a vascular assist system 100 according to one embodiment of the present invention. In some embodiments, each of the vascular assist system 100 components are implantable within a body. The vascular assist system 100 includes a vascular assist device 200 coupled to a pump 300 via a conduit 225. The vascular assist device 200 is a fluid inflatable cuff having a cover layer 220 coupled to an expandable layer 210 (FIG. 4). A cavity 250 is defined by the cover layer 220 and the expandable layer 210 (FIG. 4). The vascular assist device 200 is configured to encircle and come into contact with the outer wall of a blood vessel 20 (FIG. 7). One advantage of the embodiments of the vascular assist devices of the present invention is that the devices do not come into contact with the body blood supply (i.e., all vascular assist devices of the present invention remain outside the vasculature being augmented).

The pump 300 can be any conventional pump but is preferably a pulsatile pump. A conduit 225 (i.e., a hollow flexible tube) connects the pump 300 to the cuff 200. In an embodiment where the pump 300 is a pulsatile pump, a bladder is commonly disposed within or operably in relation to the pulsatile pump. The bladder is a flexible non-compliant or semi-compliant chamber that stores the fluid used to operate vascular assist device 200. In operation of the pulsatile pump manipulates the bladder resulting in fluid movement. In another embodiment, a reservoir (not shown) may be provided in communication with the pump. As an alternative to the bladder described above, the reservoir may be used to store fluids used by the vascular assist system 100 for the operation of the vascular assist device 200.

Operation of the pump for the activation and de-activation vascular assist device 200 is controlled by the pacing and pump controller 320. The pacing and pump controller 320 includes a programmable computer and electronics for operating the components of vascular assist system 100. Sensors 350, such as, for example, pressure sensors or electronic sensors, are positioned to detect a signal representing the cardiac cycle of a heart in a patient body. A signal representing the cardiac cycle of a heart in a patient body may be, for example, an electrical signal related to the cardiac rhythm, or the blood pressure, such as, in a blood vessel, for example, the aorta or the vena cava or pressure measured elsewhere on the patient body. A battery 310 provides power to the components of the vascular assist system 100. In the illustrated embodiment, internal coils 322 are also provided so that the battery may be charged transcutaneously.

In operation, the pacing and pump control 320 may, for example, interpret the signal representing the cardiac cycle detected by the sensors 350, execute control signals to pump 300 based on the cardiac rhythm to allow fluid into or out of the vascular assist device 200, record cardiac activity, or execute pre-programmed routines for the actuation of the vascular assist device 200. For example, to cause compression of a blood vessel, the pacing and pump controller 320 signals the pump 300 to compresses the bladder of the pump (i.e., in the case of a pulsatile pump) thereby forcing the fluid into the cuff 200 resulting in the inflation of the cuff 200. As will be described in greater detail below, the cuff 200 is positioned in relation to the blood vessel such that cuff inflation results in blood vessel compression. As will be described below, cuff activation and blood vessel compression can be advantageously synchronized with a number of parameters that are related to the cardiac cycle of a heart in a patient body.

A variety of different type of sensors may be used in vascular assist system 100 for monitoring the cardiac cycle of a heart. In one embodiment, the sensor 350 may be a pressure sensor. One suitable pressure sensor may be, for example, a pressure gage that is coupled (i.e., either integrally coupled or removably coupled) directly to the cuff 200. Alternatively, the pressure of the blood in a vessel may be measured with a pressure catheter positioned internally within the vessel. In yet another alternative, the sensor 350 may be a pressure transducer suited for measuring blood pressure within a vessel or any portion of the patient body where blood pressure may be detected and used by the system 100. A suitable pressure transducer may be either internal to or externally disposed about or within the vessel of interest. In an alternative embodiment, the sensor 350 may be an electrical sensor suited for detecting an electrical signal associated with the cardiac cycle of the heart. In some embodiments, the electrical sensor is an electrocardiogram (ECG) lead. It is to be appreciated that some embodiments of the cuff 200 comprise embodiments of the pressure sensor and/or the electrical sensor. The embodiments of the pressure sensor and/or electrical sensor may be disposed directly adjacent the cuff 200 or integrally formed in the cuff 200.

As will be described further below, an embodiment of the sensor 350 may be used to detect a signal related to the cardiac cycle of a heart. The signal is then used by the pacing and pump controller, in some embodiments, as the trigger for the activation of the cuff 200. In one embodiment, the sensor 350 is a pressure sensor and the signal related to the cardiac cycle of the heart is the pressure in a vessel. The vessel measured may also depend on the location of the cuff 200 and the desired augmentation scheme. For example, if arterial augmentation is desired, the cuff 200 will likely be implanted on the arterial side of the heart about the aorta. In this example, the pressure sensor would be disposed to measure aortic pressure. On the other hand, if venous augmentation is desired, the cuff 200 will likely be implanted on the venous side of the heart about the vena cava. In this example, the pressure sensor may be disposed to measure venous pressure in the vena cava (i.e., in either the inferior or superior vena cava) or use a measurement of arterial side pressure.

The fluid used within the vascular assist system 100 may be any of a wide variety of biocompatible fluids. The system fluid may be a liquid, such as, for example, saline, water, a glycol, such as for example, ethylene glycol. In addition the liquid may also be a mixture comprising water and a glycol or a mixture comprising saline and a glycol. The system fluid may also be a gas such as a gas that is chemically inert with the materials used to form the components in communication with the fluid. Components in communication with the system fluid include, for example, the cuff, 200, conduit 225, fluid volume compensator 1900. For example, when the cuff (i.e., layers 210 and 220) is formed from a material such as of silicone, neoprene and copolymers comprising styrene and butadiene then examples of inert gases include carbon dioxide or nitrogen. Alternatively, the system fluid may also be a gas having a density less than air. As used herein, a density less than air refers to a density less than either 1.2928 grams/liter or 0.08071 lb./cu. ft. at a standard temperature and pressure (STP) of 0 degrees C. and 760 mm Hg. Examples of suitable gases having a density less than air are helium (density of 0.1785 grams/liter or 0.01143 lb./cu. ft.); and nitrogen (density of 1.2506 grams/liter or 0.078072 lb./cu. ft.).

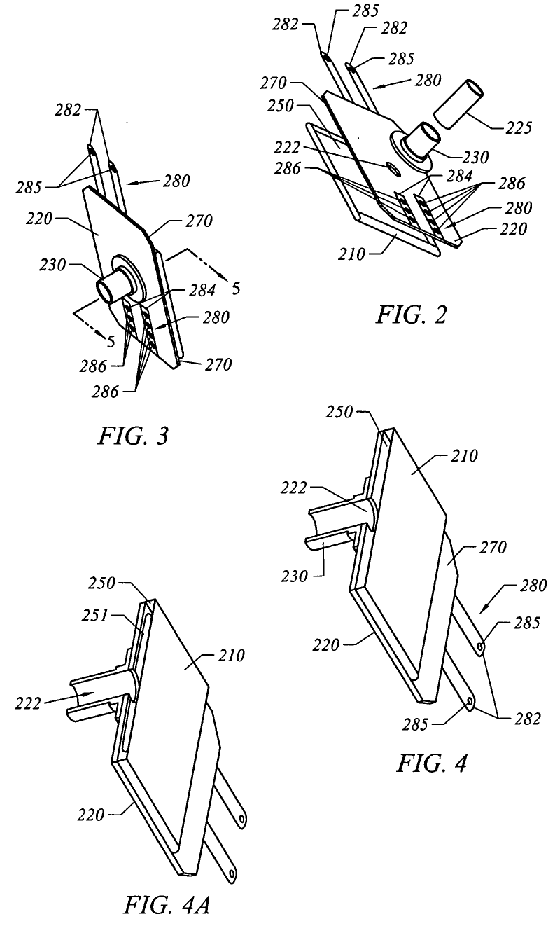

FIGS. 2, 3, and 4 illustrate a first embodiment of the ventricular assist device 200 of the present invention. The device 200 includes a compliant first layer or expandable wall 210 that is configured to be coupled to a second layer or cover layer 220 such that a cavity 250 is defined between the first layer 210 and the second layer 220 (FIGS. 3 and 4). The second layer or cover layer 220 includes an opening 222 for fluid access to the cavity 250, mechanical connection for fluid system via connection 230, a semi-rigid support base for cavity 250 and expandable wall 210 and mechanical support for the fasteners and/or cuff closure system 280 FIGS. 2, 3, 4 and 7).

In some embodiments, the first layer 210 is coupled to the second layer 220 about a perimeter of the first layer 210. In other embodiments, the first layer 210 is coupled to the second layer 220 about a portion of the perimeter of the second layer 220. In another embodiment, a perimeter of the second layer 220 extends beyond the perimeter of the first layer 210. The expandable layer 210 and cover layer 220 could also be thought of, relative to the vasculature, as in inner layer (expandable layer 210) and an outer layer (cover layer 220). Alternatively, the inner layer 210 can be coupled to the outer layer 220 about a perimeter of the inner layer 210. In another embodiment, a perimeter of the outer layer 220 extends beyond the perimeter of the inner layer. Alternatively, the outer layer 220 can include a first edge, a second edge, a third edge and a fourth edge. At least one of the edges can be collocated with an edge along the perimeter of the inner layer 210.

The cover layer or second layer 220 includes a length and a width and the first layer or expandable layer 210 also includes a length and a width. In some embodiments of the device 200, the length of the first layer 210 is less than the length of the second layer 220. In another embodiment of the device 200, the width of the first layer 210 is less than the width of the second layer 220. In another embodiment, the length of the first layer 210 is sufficient for the first layer 210 to partially completely encircle a portion of a blood vessel. The length of the first layer 210 may be long enough to partially encircle, for example, a portion of the ascending aorta, the descending aorta, the superior vena cava, the inferior vena cava or a portion of a blood vessel that also includes a set of intercostal arteries or a set of intercostals veins.

In another embodiment, the length of the second layer 220 is sufficient for the second layer 220 to completely encircle a portion of a blood vessel. The second layer 220 may also include a fist end and a second end. When the second layer 220 is configured to completely encircle a portion of a blood vessel, the first end and the second end of the second layer overlap. The length of the second layer 220 may be long enough to encircle, for example, a portion of the ascending aorta, the descending aorta, the superior vena cava, the inferior vena cava or a portion of a blood vessel that also includes a set of intercostals arteries or a set of intercostals veins. The length of the second layer 210 is configured to partially encircle a blood vessel when installed about a blood vessel.

The cover layer 220 also includes at least one opening 222 in fluid communication with the cavity 250 (FIGS. 2 and 4). The cuff 200 includes a port 230 that can be coupled to the conduit 225 to deliver fluid to the cavity 250. The second layer 220 defines an opening 222 to provide fluid access to the cavity 250. A coupling 230 is provided to couple the conduit 225 to the opening 222 in the second layer 220 (FIGS. 2 and 4). The conduit 225 is coupled to the second layer or cover layer 220 in communication with the opening 222. The conduit 225 is configured to be coupled to the pump 300. As such, the conduit 225 and the fluids therein are in fluid communication with the cavity 250. In response to fluid pressure changes and/or volume changes of the cavity 250, the compliant first layer 210 is configured to deform (i.e., expand in response to increasing pressure or volume of the cavity 250). When the vascular assist device 200 is installed about a blood vessel (i.e., FIG. 7), the first layer 210 at least partially encircles the blood vessel. The pump and pacing controller 320 directs the pump 300 to supply fluid to the device 200 in response to and in synchronization with a signal representing the cardiac cycle of a heart in a patient body. Fluid then enters the cavity 250 causing it to increase in volume and/or pressure thus deforming the expandable wall 210. As the first layer 210 deforms (under pressure of the expanding cavity 250), the vessel encircled by the cuff 200 is compressed and blood within the vessel is urged onward. As such, the fluid (i.e., the gas or the liquid) is configured to be selectively communicated in synchronization with the cardiac cycle to the cavity 250 via a conduit 225 in communication with the opening 222 in the cover layer 220.

Embodiments of the vascular assist device of the present invention provide a compliant first layer 210 that is configured to engage internal vasculature. The second layer or cover layer 220 is coupled to the first layer 210 defining a cavity 250. The second layer 220 has a stiffness greater than a stiffness of the first layer 210. In response to changing volume of cavity 250, the first layer is configured to be deformed in response to a change in the volume of the cavity 250. Additionally, the first layer 210 is deformable such that when the pressure inside the cavity 250 increases, the first layer 210 deforms (i.e., expands). The second layer or cover layer 220 is configured to be flexible enough to encircle a blood vessel however, rigid enough not to deform under the range of pressures and volumes experienced by the cavity 250. Through the advantageous selection of the flexibility of the cover layer 220 and the expandable layer 210, the changes in fluid pressure or cavity volume are more likely to deform the expandable wall 210 and result in compression of the vessel of interest.

The advantageous functioning the cover layer and the expandable layer may be accomplished, for example, through selection of the materials selected for each of the layers. The expandable layer material may be selected to have a stiffness less than the stiffness of the cover layer. The expandable layer 210 may be fabricated with a first material and the cover layer 220 may be fabricated with a second material. In some embodiments, the first material is a first silicone elastomer and the second material is a second silicone elastomer. The first silicone elastomer may be a 5-50 A silicone elastomer having a minimum of 500% elongation. The second silicone elastomer is a 65-95 A silicone elastomer having less than a 400% elongation. In an alternative embodiment, the first material may be an elastomer having a hardness of 5-50 shore A and a minimum elongation of 500%. The second material may be an elastomer having a hardness of 65-95 shore A and a maximum elongation of 400%.

To maximize the efficiency of the device 200, the cover or second layer 220 is configured to be flexible, but does not stretch or expand under the pressure inside the cavity 250. The first layer or inner layer 210 is made of a more flexible (i.e., less stiff) material than the cover layer 220. In one particular embodiment, the inner wall or first layer 210 can be made of a 5 to 50 A silicone elastomer with a minimum of 500% elongation and the outer or cover layer 220 can be made out of less compliant silicone such as a 65 to 95 A silicone elastomer with less than 400% elongation. The first and second layers may, for example, be formed from a material that is one of silicone, neoprene and copolymers comprising styrene and butadiene. In some embodiments, the outer layer 220 is fabricated in the same manner as the first layer 210 and can be attached to the inner layer 210 by adhesives such as silicone RTV. The outer layer 220 can also be over-molded on the inner layer 210 by insert molding.

Other suitable materials for the cuff 200 (i.e., suitable materials for the layers 210 and 220) include C-Flex™, santoprene, Kraton™, PVDF, etc. Possible fabrication methods include injection molding, casting, dip molding, insert molding, over molding and blow molding. Kraton™ and C-Flex™ refer generally to thermoplastic elastomers (TPE's) that are copolymers of styrene, butadiene, and other polymers which range in hardness from 5 shore A durometer to 95 shore A durometer. C-Flex™ is commercially available from, for example, Consolidated Polymer Technologies, Inc. (CPT) of Clearwater, Fla. Kraton™ is commercially available from, for example, GLS Corporation of Delaware. Both Kraton™ and C-Flex™ are desirable materials because of their high bio-compatibility, high modulus of elasticity, and easy fabrication.

To improve the performance and durability of the cuff 200, the layers 210, 220 and other components in vascular assist system 100 may each be reinforced by an additional material or a reinforcement element. Reinforcement, as used herein, includes the addition of a reinforcing element to a material to prevent rupture, prevent crushing, or adjust the material properties of the material. Examples of how reinforcing elements may be used to alter the material properties of a material include the addition of reinforcing elements to alter the elongation properties of a material, reduce the permeability of a material or improve the strength of a material. In one illustrative embodiment, the second layer or cover layer includes a reinforcement element. The reinforcement element is coupled to the cover layer and configured such that the reinforcement element maintains the length and width of the cover layer as fluid is ported into and out of the cavity 250. As such, the reinforcing element is used to maintain the rigidity of the cover layer 250 so that the desired deformation of the layer 210 occurs. In this regard, the cover layer 250 provides mechanical strength for the advantageous deformation of the expanding layer 220.

In addition, the reinforcing element or elements may be incorporated into the material such that material reinforcement is selective and adjustable. Representative reinforcing materials include polyester, nylon, para-aramid fiber, stainless steel, platinum, superelastic nitinol and alloys of nickel and titanium. The para-aramid fiber may be commercially available, such as, for example, Kevlar™, and/or polyester fibers. Alternatively, reinforcement may accomplished by simply adjusting the wall thickness a component to that the thicker wall portions of the component act as reinforcing elements. The conduits 225, 228 may also employ reinforcing elements so that the walls of the conduit do not collapse under pressure of tissue growth within the body.

The use of fiber reinforcement elements for the cover layer and/or expandable layers 210, 220 of the device 200 may also reduce the permeability of the layers 210, 220, thus reducing fluid loss through the walls. Additionally, to minimize fluid loss of the vascular assist system 100 the surfaces of the pump 300, cuff 200 and conduit 225 in contact with the fluid used in the system 100 may be coated with impermeable or semi-permeable materials such as polyethylene, polypropylene, etc. Alternatively, the inside surfaces (i.e., surfaces not in direct contact with the patient body) and/or outside surfaces (i.e., surfaces in direct contact with the patent body) of embodiments of the cuff 200, pump 300, conduits 225, 228 and the fluid volume compensator 1900 may be coated with impermeable or semi-permeable materials such as polyethylene, polypropylene, etc. to reduce fluid loss from the system 100. Metallic powder coatings can also be used for the same purpose.

The cover layer or second layer 220 extends beyond the chamber or cavity 250, thereby creating a flexible overlapping set of flaps 270. As described above the cover layer 220 provides an opening 222 and mechanical support for the attachment of coupling 230. In some embodiments of the vascular assist device 200, the cover layer 220 also provides the mechanical attachment point for the fastening means 280 used to secure the vascular assist device 200 about a portion of a vessel. In other embodiments, the vascular assist device 200 is configurable between an uninstalled configuration (i.e., when the fastening means 280 are not coupled, FIGS. 2, 3 and 4) and an installed configuration when the fastening means 280 are coupled (i.e., FIG. 7). In the illustrated embodiments, the cuff 200 is configurable between a first, planer configuration (FIGS. 2, 3 and 4) and a second configuration in which it is tubular or oval in shape and configured to be positioned around a blood vessel (i.e., a portion of the ascending aorta 20 as in FIG. 7). It is to be appreciated that other embodiments of the vascular assist device 200 are possible where both the first and second configurations are generally tubular and the difference between the first and second configurations depends on whether or not the fastening elements are coupled (second configuration) or uncoupled (first configuration).

The device 200 is held in position about a vessel by fastening elements 280. The flaps 270 can support the fastening elements 280 for the device 200 (FIGS. 2, 3 and 4). The fastening elements 280 have cooperatively configured ends 282 and 284. In the illustrated embodiment, one end 282 has a feature 285 configured to be cooperatively coupled to one of the plurality of features 286 on end 284. When the device 200 is configured about a vessel, the ends 282, 284 may be adjustably and repeatably fastened. The device 200 is adjustably fastened because the feature 285 on end 282 may be coupled to any one of the features 286 depending upon the size (i.e., external diameter) of the vessel. The device 200 is repeatably fastened because the cooperative fastening elements 285, 286 may be coupled and uncoupled repeatably. The embodiments of the vascular assist device having the adjustable and repeatable features may advantageously be employed for a wide variety of vessel sizes (i.e., diameter). A physician implanting the device 200 may install (i.e., secure about a vessel of interest) and test (i.e., activate the device by porting and removing fluid from the cavity 250) the device in a number of different configurations and positions to ensure proper fit and operation.

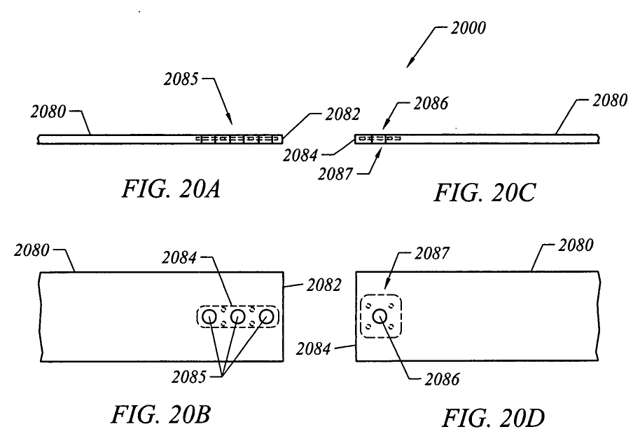

Another aspect of the adjustable quality of the fastening elements 280 is that independent attachment of the ends 282. Independent attachment refers to the ends 282 not being coupled to a correspondingly located feature 286. By reference to FIG. 2, independent attachment means that one end 282 may be attached to a feature 286 near the port 230 while the other end 282 may be attached to a feature 286 near the edge of the layer 220. Note that the left side has three attachment features 286 while the right side has four attachment features 286 with a different spacing between each attachment feature 286. The variability of the attachment features underscores the configurability of the independent attachment feature of fastening elements 280. The independent attachment feature provides an additional dimension of configurability to embodiments of the device 200. It is to be appreciated that by changing or adjusting to which of features 286 the ends 282 attach the device 200 may be configured into a wide array of shapes, such as, generally cylindrical with an adjustable diameter, or variously sized truncated conical shapes having adjustable base and apex diameters. FIGS. 2, 3 and 4 illustrate one embodiment of a fastening element 280 for discussion purposes. Additional embodiments of the fastener elements 280 and different types of fastening are described in greater detail below with regard to FIGS. 20A-29.

In an alternative embodiment, a bladder 251 can be inserted or formed into the cavity 250 (FIG. 4A). The bladder 251 would be attached to the inside wall of the cover layer 220 so as to be in fluid communication in the with the fluids in the system and the pump 300 (i.e., via an opening 222). When the bladder 251 is present, fluids are ported into and out of the bladder 251 that in turn expands (preferentially instead of the cover layer 220) and deforms the expanding layer 210 into contact with the vessel. One advantage of using the bladder 251 is that the expanding layer can be used as a safety device. Should any leaks occur in the bladder 251, leaking fluid would be captured in the cavity 250 created by the expanding layer 210 and cover 220. As a result, system fluids leaking out of the bladder 251 would not enter the patient body. The bladder 251 could be formed from the same or similar materials as the layers 210, 220 and possess the advantageous characteristics described above with regard to, for example, selective deformation of the layer 210.

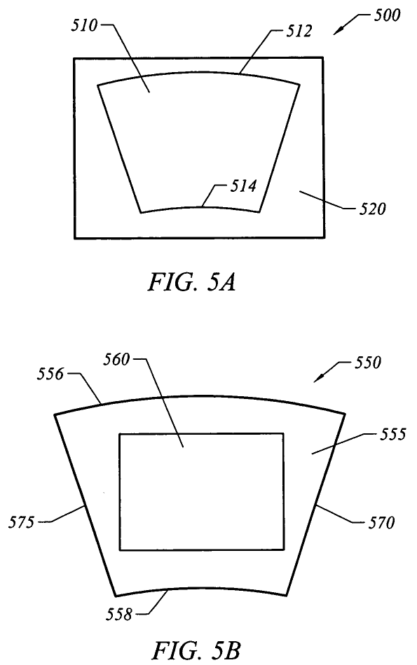

In the embodiments illustrated in FIGS. 2, 3 and 4, the cover layer 220 and the expandable layer 210 each have a generally rectangular shape with the cover layer 220 being larger than the expandable layer 210. It is to be appreciated that in other alternative embodiments of the vascular assist device 200 of the invention the cover layer 220 and expandable layer 210 may have other shapes. In some embodiments, the cover layer 220 and the expandable layer 210 will have different shapes. FIGS. 5A and 5B illustrate alternative embodiments of vascular assist devices of the present invention. FIG. 5A illustrates a vascular assist device 500 having a cover layer 520 and an expanding layer 510. The cover layer 520 has a generally rectangular shape while the expanding layer 510 has a generally trapezoidal shape. FIG. 5B illustrates a vascular assist device 550 having a cover layer 555 and an expanding layer 560. The cover layer 555 has a generally trapezoidal shape and the expanding layer 560 generally rectangular shape.

The vascular assist devices 500 and 550 may also represent how embodiments of the device of the present invention may be modified to, for example, more readily engage and augment a variety of vessel types. The vascular assist device 500 illustrates a rectangular cover layer 520 that may be an advantageous shape from the standpoint of ease for fastening the device 500 about the vessel (FIG. 5A). The expandable layer 510 has a trapezoidal shape having a base 512 and an apex 514. The trapezoidal shape may advantageously augment curved vasculature such as, for example, the ascending aorta.

The trapezoidal shape may also be used to further enhance the blood flow augmentation. The vascular assist device 500 may be coupled to the fluid conduit (not shown) in a manner such that fluid enters the cavity 250 proximate to the apex 514 and then propagates towards the base 512. In this manner, when the vascular assist device 500 is coupled to a vessel of interest, the device 500 may be positioned so that the augmentation direction of the device (i.e., from apex 514 towards base 510) is aligned with the direction of fluid flow in the vessel. As such, the vascular assist device 500 may be coupled to a vessel of interest in such a way that the fluid movement resulting from augmentation is in a direction from the apex 514 towards the base 512. 1000811 Alternatively, the vascular assist device 500 may be coupled to the fluid conduit (not shown) in a manner such that fluid enters the cavity 250 proximate to the base 512 and then propagates towards the apex 514. In this manner, then the vascular assist device 500 is coupled to a vessel of interest, the device 500 may be positioned so that the augmentation direction of the device (i.e., from base 510 towards apex 514) is aligned with the direction of fluid flow in the vessel. As such, the vascular assist device 500 may be coupled to a vessel of interest in such a way that the fluid movement resulting from augmentation is in a direction from the from base 510 towards apex 514.

The vascular assist device 550 also illustrates how the shape of the cover layer 555 may shaped to be more easily engaged with the vessel of interest (FIG. 5B). The cover layer 555 has a trapezoidal shape with a base 556 and apex 558. The trapezoidal shape is useful in providing a wide array of non-cylindrical shapes when the edges 570 and 575 are joined together about the vessel of interest. Rectangular and trapezoidal shapes have been used with the illustrative embodiments in FIGS. 5A and 5B only to illustrate these additional advantages and highly configurable nature of the vascular assist devices of the present invention. Both the cover layer and the expanding layer may have other shapes, such as oval, elliptical, polygonal or irregular shapes to achieve the vessel engagement and augmentation features described above.

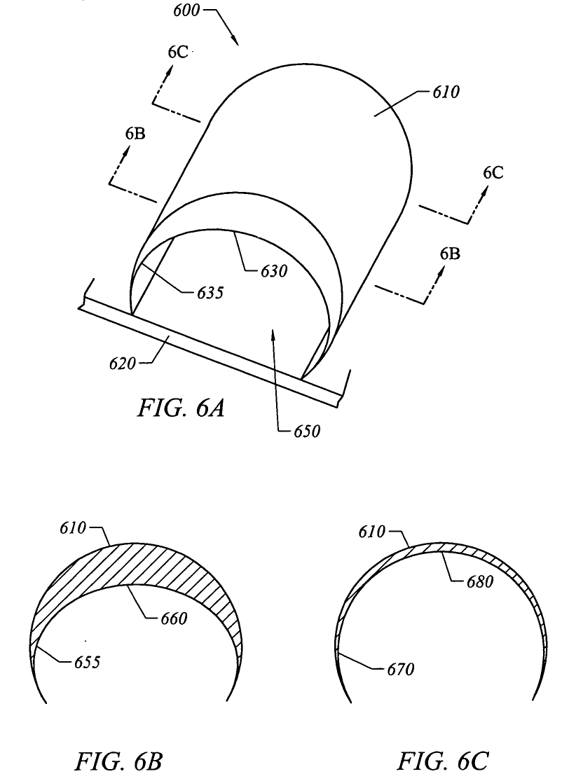

FIGS. 6A, 6B and 6C will now be used to describe an additional feature of embodiments of the expandable wall according to the present invention. FIG. 6A illustrates a device 600 having an expandable wall 610 and cover layer 620 that define a cavity 650. The wall thickness of the expanding layer may be advantageously adjusted and controlled so that the expansion of the cavity 650 is controllable and selectable. The expansion characteristics of cavity 650 is one factor used to vary the activation response and operation of the device 600. Because the expanding layer 610 is designed to deform in response to increasing pressure within the cavity 650, the physical properties and yield limits of the expanding wall may be advantageously used to control the deformation of the expanding wall 610. In this manner, the expandable wall 610 is said to be selectably deformable. By selectively controlling the deformation of the expanding wall 610, the response time, augmentation efficiency and other operational characteristics of the device 600 may be controlled and optimized.

The variation of radial thickness (i.e., “sidewall” thickness) and the longitudinal thickness will now be described with regard to the vascular assist device 600. In the view of FIG. 6A, the proximate end of the expanding wall 610 has a radial thickness 635 and a longitudinal thickness 630. The distal end of the expanding wall 610 (section view FIG. 6B) has a radial thickness 655 and a longitudinal thickness 660. At a point between the proximal end and the distal end (section view, FIG. 6C) the expanding wall 610 has a radial thickness 670 and a longitudinal thickness 680. The device 600 has a variable, selectable longitudinal deformation because longitudinal thickness 630 is about the same as longitudinal thickness 660 but both are greater than the longitudinal thickness 680. As such, as the pressure in the cavity 650 increases, the longitudinal deformation of the expanding wall 610 will occur first in the region adjacent longitudinal thickness 680. In other words, the longitudinal deformation of the expanding wall 610 will propagate from the mid-region near longitudinal thickness 680 towards the proximal and distal ends (i.e., longitudinal thickness areas 630, 660). The expanding wall 610 is also selectively radially deformable. The device 600 has a variable, selectable radial deformation because radial thickness 635 is about the same as radial thickness 655 but both are greater than the radial thickness 670. As such, as the pressure in the cavity 650 increases, the radial deformation of the expanding wall 610 will occur first in the region adjacent longitudinal thickness 670 (i.e., the thinner radial thickness). In other words, the longitudinal deformation of the expanding wall 610 will propagate from the mid-region near longitudinal thickness 670 towards the proximal and distal ends (i.e., radial thickness areas 635, 655).

While the device 600 of FIGS. 6A, 6B and 6C has selected both radial and longitudinal deformation to occur centrally first, other selective deformation configurations are possible. For example, some device embodiments of the invention may have only radial or only longitudinal deformation. Other embodiments may have selective radial deformation occurring in a first portion of the expanding wall while selective longitudinal deformation occurs in a second different portion of the expanding wall. Radial and longitudinal thickness may be adjusted independently or cooperatively to provide a wide array of selective deformation configurations. While the selective deformation has been described using wall thickness, it is to be appreciated that other methods of providing selective deformation of the expanding wall 610 are possible. One other method for providing selective deformation is the advantageous positioning of a reinforcing element on or in the expanding wall. The use and types of suitable reinforcing elements are described elsewhere. As such, reinforced portions may not deform as quickly or under the same reassure ranges as the unreinforced portions. Thus, altering the reinforcement configuration of the expanding wall 610 may also be used to provide the selectively deformable feature of embodiments of the expanding wall 610 of the present invention. While described with regard to a representative vascular assist device 600, it is to be appreciated that the advantages of the selectively deformable expanding wall 610 may be used with the expanding wall utilized in any of the other vascular assist device embodiments of the present invention.

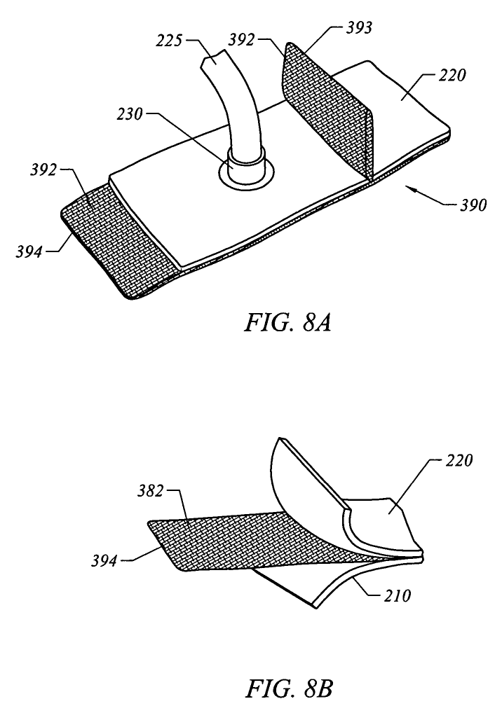

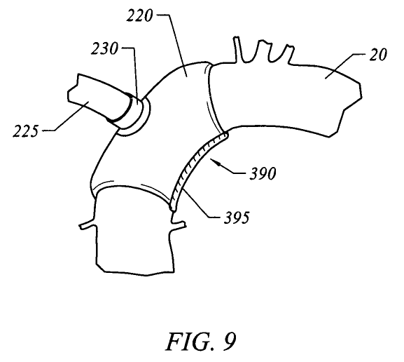

Alternative fastening means 810 are possible. For example, a fabric layer 392 may be incorporated into a vascular assist device 390 and then sutured together as the fastening means for securing vascular assist device 390 in place about a vessel (FIGS. 8A, 8B and 9). The vascular assist device 390 is similar in all respects to the embodiments of the vascular assist 200 described above and like reference numbers have been used. A fabric layer 392 is incorporated into the vascular assist device 390 between the cover layer 220 and the expandable layer 210 as illustrated in FIG. 8B. The fabric layer 392 includes an end 394 and a looped end 393. The fabric layer 392 may have a thickness on the order of a few microns and can be fabricated from a material such as PTFE, nylon or polyester. When the vascular assist device 390 is positioned about a vessel, the end 394 and the looped end 393 are sutured together thereby securing the vascular assist device 390 in place. In this way, suturing in another fastening means that may be used to secure a vascular assist device embodiment about a vessel. Turning to FIG. 8, the vascular assist device 390 is fastened about the ascending aorta 20 using suture 395.

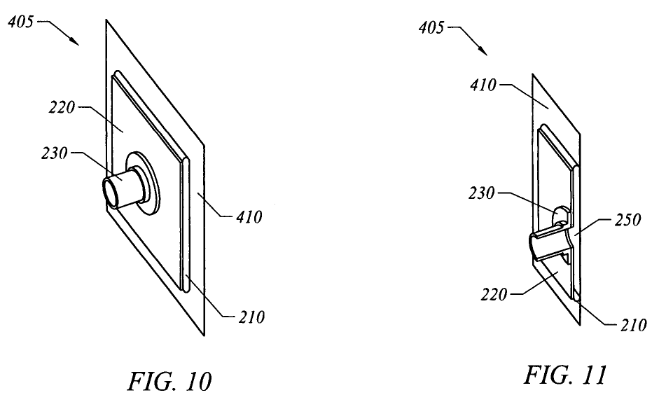

The embodiments of the vascular assist device of the invention have thus far been described where the expanding layer 210 is in direct contact with the vessel to be augmented by the vascular assist system 100. Depending on a number of factors such as, for example, vessel wall strength and the patients' physiology, there may be circumstances when another layer could be used to protect the vessel wall by being positioned between the expanding layer 210 and the vessel wall. In some instances, the patient's vessel wall health may be less than optimal or a physician may want additional protection of the vessel from the augmentation activity of the device. In either case and for perhaps other reasons, embodiments of the vascular augmentation system 100 also provide a vascular engaging layer that is disposed between the expanding layer 210 and the vessel wall. The vascular assist device 405 is one embodiment of a vascular assist device of the invention that provides a vessel wall protection feature (FIGS. 10, 11 and 12). The vascular assist device 405 is similar to the other vascular assist device embodiments described above. The vascular assist device 405 also includes a vascular engaging layer 410 positioned adjacent to the expandable layer 210. The a vascular engaging layer 410 is larger than both the expandable layer 210 and the cover layer 220. The vascular engaging layer 410 is bonded, affixed or other wise joined to the expandable layer 210 such that the vascular engaging layer 410, the expandable layer 210 and the cover layer 220 form a unitary structure. For example, the vascular engaging layer 410 may be insert-molded to the inner layer 210. Alternatively or additionally, a primer may be applied to improve the adhesion of the vascular engaging layer 410 to the inner layer 210. The vascular engaging layer 410 can have a thickness on the order of a few microns and can be fabricated from a fabric-type material such as PTFE, nylon or polyester. The vascular engaging layer 410 may be a graft layer.

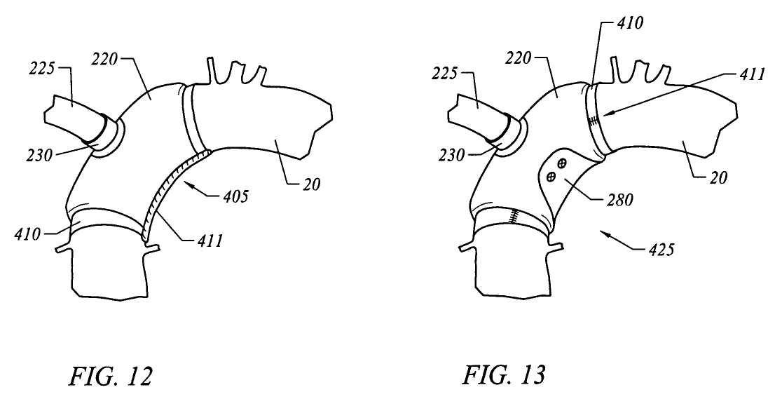

The vascular engaging layer 410 is sufficiently long to encircle a vessel (i.e., the aorta or the vena cava). As illustrated in FIG. 12, when the vascular assist device 405 is positioned about a vessel, the vascular engaging layer 410 encircles the vessel 20 and is sutured 411 together. As such, the vascular assist device 405, like the vascular assist device 390, employs sutures as the fastening means to secure the vascular assist device in place about the vessel of interest. While the vascular assist device 405 illustrates an embodiment where the vascular engaging layer 410 is integrally formed to the layer 210, it is to be appreciated that the vascular engaging layer 410 may advantageously employed with the other embodiments of the vascular assist device 200. As illustrated in FIG. 13, a vascular assist device 200 is shown secured in place (i.e., fastener means 280) about the ascending aorta 20. Before the vascular assist device 200 was installed about the aorta 20, a vascular engaging layer 410 was first fastened about the ascending aorta 20 using sutures 411. FIG. 13 illustrates the vascular engaging layer 410 or graft sutured in place around the vessel but the cover layer provides a fastening element 280 to secure the cover layer 220 about the vessel 20 thereby securing the device 425 about the vessel 20. It is to be appreciated that the vessel engaging layer 410 or graft layer may be a separate piece from the cuff 200 (FIG. 13) or may be integrally formed with a cuff by coupling it to the expanding wall (FIG. 12). Thus, an embodiment of the vascular engaging layer 410 may be used with any of the vascular assist embodiments of the present invention to achieve the vessel protection feature described above.

The embodiments of the vascular assist device of the invention thus far have included continuous cuff shapes that are particularly suited to engaging and augmenting vessels having few or no protubewrances or tributary vessels attached. Segmented cuffs, however, may be advantageously utilized to augment vessels having naturally occurring or artificially implanted vessels attached. Examples of naturally occurring vessels are the descending aorta with arterial intercostals and the vena cava with venous intercostals. An example of an artificially implanted vessel is the ascending aorta with a bypass graft attached thereto. In each of these cases it is desirous to augment the main vessel (i.e., aorta or vena cava) without harm to the attached vasculature (i.e., intercostals or bypass graft). The embodiments of the segmented cuffs of the present invention provide the advantages of the earlier described cuff embodiments with the added benefit of providing configurable augmentation to reduce or eliminate harm to naturally or artificially attached vasculature.

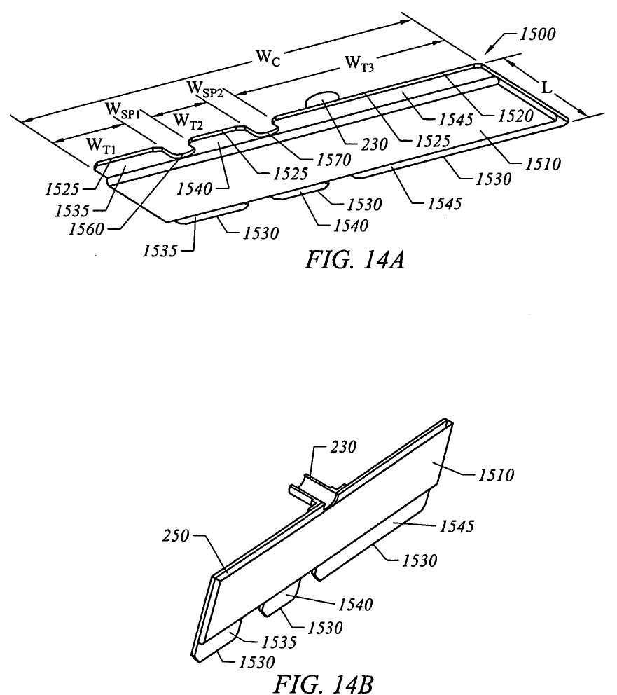

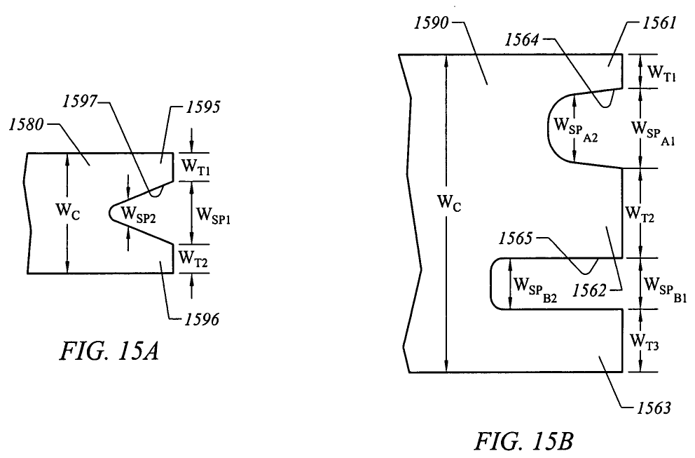

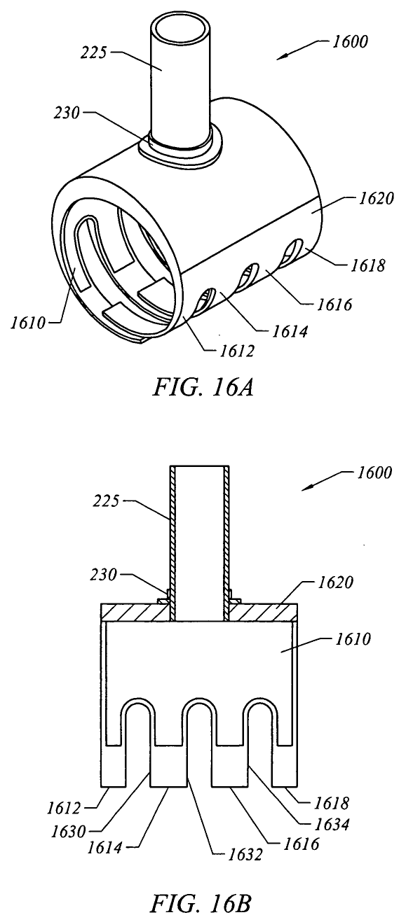

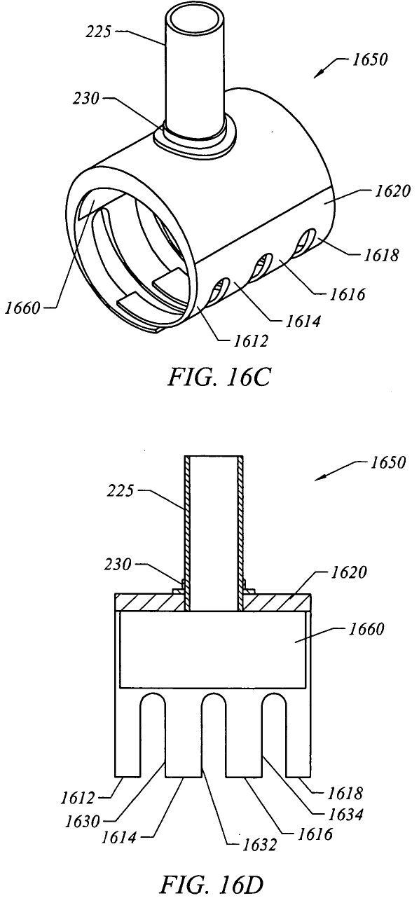

Embodiments of the segmented cuff of the present invention will now be described with regard to FIGS. 14A, 14B, 15A, 15B and 15C. The segmented cuff 500 of the present invention is configured similar to the earlier cuff embodiments with regard to the material selection for the cover and expanding layer, fastening elements and fluid connections. The segmented cuff 500 is segmented in that it includes openings or cutouts between the tabs. The specific shape of the cutout is referred to herein as the tab spacing profile. The tab spacing profile is used to configure the segmented cuff such that the cuff may wrap around a vessel of interest while not harming or obstructing flow into naturally occurring or artificially implanted vessels. Additionally, the segmented portions may also be used to avoid protuberances or other obstacles along the length of the vasculature to which the cuff 500 is attached. These openings or tab shape profiles are defined on opposing sides of the segmented cover layer 1520. The tab shape profiles are configured as notches or recesses defined along the opposing edges 1525 and 1530 of the segmented cover layer 1520. It is to be appreciated that embodiments of the segmented cuff as possible where the expandable layer 1510 is also segmented as described below with regard to FIG. 16B. In an embodiment in which the edges of the inner 1510 and outer 1520 segmented layer are coterminous, the openings or tab spacing profiles are defined in both the inner 1510 and outer 1520 segmented layers. Of course, the layers are sealed to maintain the integrity of the cavity 250 formed there between.

Returning to FIG. 14A, the segmented cuff 1500 includes a segmented cover layer 1520 and an expandable layer 1510 that are structurally and operationally similar to the cover layer 220 and expandable layer 210 described in other cuff embodiments. The segmented cover layer includes a first end 1525 and a second end 1530. The first end 1525 and the second end 1530 each have at least two tabs (i.e., 1535, 1540 and 1545). In the illustrative embodiment of FIG. 14A, three tabs (i.e., 1535, 1540 and 1545) are shown. Each of the tabs (i.e., 1535, 1540 and 1545) has a width. The sum of the widths of all the tabs (i.e., 1535, 1540 and 1545) on one end (either end 525 or 530) is less than the width of the segmented cover layer 1520. At least two tabs on the first and second ends are configured to be removable coupled such that the segmented cuff is reconfigurable between a first configuration in which the at least two tabs on the first and second ends are separate and a second configuration in which the at least two tabs on the first and second ends are coupled. Any of the fastening elements described above or below may be provided on segmented cover layer 1520 to removeably couple the first and second ends 1525, 1530.

Another feature of the segmented cuff 1500 is the advantageous use of tab spacing profiles to further accommodate naturally occurring or artificially implanted vessels. Tab spacing profiles (1560 and 1570) have a width and are used to describe the spatial relationship between adjacent tabs. A tab spacing profile is used to describe the distance between the adjacent tabs (i.e., spacing profile width) and the shape of the notches formed by the tab profile between adjacent tabs. The tab spacing profile may be used to configure the resulting segmented cuff shape when the segmented cuff is implanted about a vessel. When the segmented cuff 1500 is installed about a vessel, the illustrative tab spacing profiles 1560 and 1570 will produce elongate rectangular segmented spaces to accommodate naturally occurring or artificially implanted vessels. It is to be appreciated that numerous tab spacing profiles are possible to accommodate a wide variety of vessel sizes and configurations. For any segmented cuff configuration the width of the segmented cuff is the sum of the widths of each of the tabs and the widths of the tab spacing profiles. For example, the width of segmented cuff 1500 is equal to the sum of the width of tabs 1535, 1540 and 1545 and the width of tab spacing profiles 1560 and 1570. The representative embodiment of FIG. 14A also illustrates how a variety of tab widths may be utilized in a segmented cuff. As illustrated, tab 1545 is much wider than tabs 1535 and 1540. The representative embodiment of FIG. 14A also illustrates the use of two similar tab spacing profiles. Tab spacing profile 1560 between tab 1535 and tab 1540 is the same a the tab spacing profile 1570 between tab 1540 and tab 1545.

The illustrative embodiments of FIGS. 15A, and 15B illustrate the configurability of the tabs and tab spacing profiles advantageously employed by embodiments of the segmented cuff of the present invention. In FIG. 15A, an alternative segmented cover layer 1580 provides a non-uniform tab spacing profile in contrast to the uniform tab spacing profiles 1560 and 1570 (FIG. 14A). As used herein, a uniform tab spacing profile has a uniform width throughout its length. Accordingly, tab spacing profile 1597 in non-uniform because the first tab spacing profile width (Wₛₚ₁) is greater than the second tab spacing profile width (Wₛₚ₂). When installed about a vessel, the tab spacing profile of the illustrative segmented cuff 1580 will provide diamond shaped segments to accommodate naturally occurring or artificially implanted vessels. The widths (W_{T1} and W_{T2}) of each of the tabs 1595 and 1596, respectively, may also be modified and need not be equal as in the illustrative embodiment of FIG. 15A.

The illustrative segmented cuff embodiment of FIG. 15B further illustrates the configurability of segmented cuffs of the present invention. The segmented cover layer, 1590 illustrates three different tab widths and two different tab spacing profiles. Segmented cover layer 1590 includes a first tab 1561, a second tab 1562 and a third tab 1563. The second tab 1562 is wider than both the first tab 1561 and the third tab 1563 (i.e., Wₜ₂>Wₜ₁ and Wₜ₂>Wₜ₃). The third tab 1563 is wider than the first tab 1561 (Wₜ₃>Wₜ₁). Additionally, there is illustrated two different tab spacing profiles. Tab spacing profile 1564 is non-uniform (i.e., Wₛₚₐ₁>Wₛₚₐ₂) and has a shape that will produce a more rounded diamond shape segment than the tab spacing profile 1597 of FIG. 15A. Tab spacing profile 1565 is uniform (i.e., W spb1=W spb2) and will result in rounded elongate segments larger than the rounded elongate or circular segments provided by the tab spacing profiles 1560 and 1570 of FIG. 14A. As such, tab spacing profile 1565 is advantageously capable of accommodating naturally occurring or artificially implanted vessels of a larger diameter. Alternatively, the tab spacing profile 1565 may also be useful because the larger elongate rectangular segments provides greater variability for the location of the naturally occurring or artificially implanted vessels along the vessel of interest. Thus, the segmented cover layer 1590 illustrates an embodiment of a segmented cuff of the present invention having at least two different tab spacing profiles (i.e., 1564 and 1565).

The above illustrative embodiments of the segmented cuffs of the present invention have described the cover layer as related the tabs and tab spacing profiles. FIGS. 16A-16D will provide additional details regarding the various configurations of the expandable layer embodiments utilized in segmental cuffs of the present invention. FIGS. 16A and 16C are an illustrative embodiment of the segmented cuffs 1600 (FIGS. 16A and 16B) and 1650 (FIGS. 16C and 16D). The illustrated segmented cuffs 1600, 1650 have a cover layers 1620 and expandable layers 1610 and 1660 that are structurally and operationally similar to the embodiments of the cover layer 220 and expandable layer 210 described above. Segmented cuffs 1600 and 1650 includes tabs 1612, 1614, 1616 and 1618 that define tab spacing profiles 1630, 1632 and 1634. In these illustrative embodiments, the segmented cuff 1600, 1650 provide tabs having two different widths (i.e., tabs 1614 and 1616 are equal and wider than the tabs 1612 and 1618). Even though the tabs have different widths, the tab spacing profiles 1630, 1632 and 1634 are equal. The segmented cuffs 1600, 1650 provides advantageous elongated rectangular tab spacing profiles while also using different cuff widths to provide better segment alignment for the naturally occurring or artificially implanted vessels along the vessel of interest.

The primary difference between the segmented cuffs 1600 and 1650 is the configuration of the expandable layers 1610 and 1660. The expandable layers 1610 and 1660 are best described through reference to FIGS. 16B and 16D. Expandable layer 1610 is a segmented expandable layer because it conforms generally to the widths of the tabs 1612, 1614, 1616 and 1618 as well as the tab spacing profiles 1630, 1632 and 1634. Expandable layer 1660 is a continuous expandable layer because it maintains its shape without regard to the width of the tabs or tab spacing profile in the segmented cuff 1650. As such, segmented cuff embodiments of the present invention may have either segmented expandable layers (i.e., layer 1610) or continuous expandable layers (i.e., layer 1660).

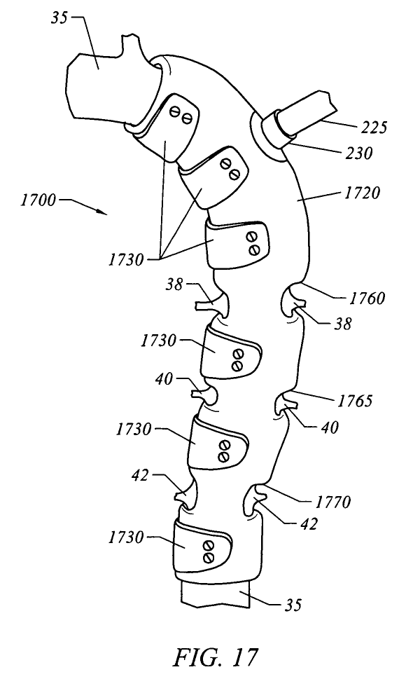

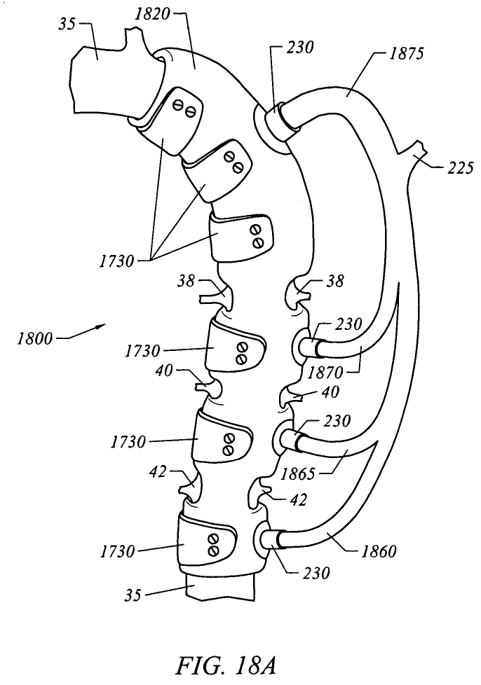

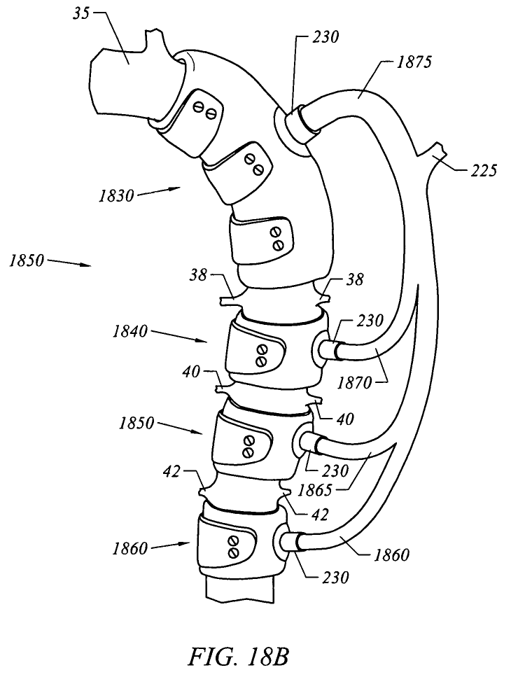

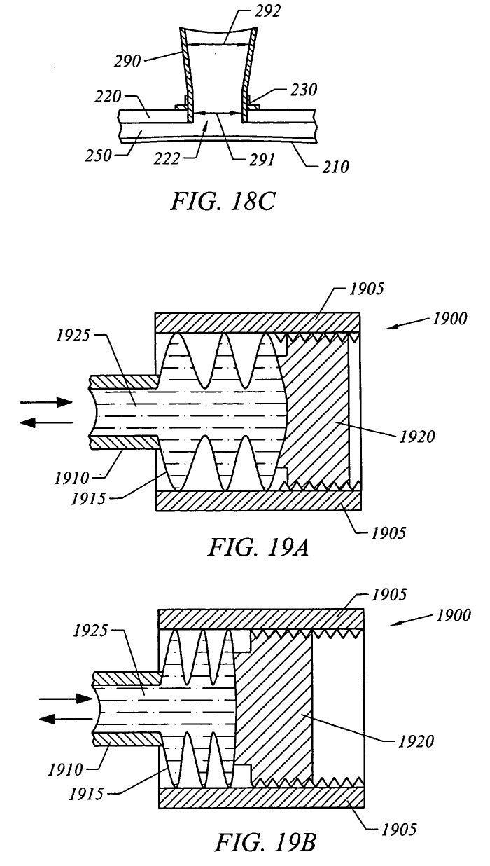

Additional advantages of the segmented half embodiments of the present invention will be appreciated with reference to FIGS. 17, 18A and 18B. The segmented cuff embodiments 1700, 1800, and 1850 provide additional details regarding the configurability of the segmented cuff of the present invention and the ability to accommodate naturally occurring or artificially implanted vessels along the vessel of interest. While the applicable to artificially occurring vessels (i.e., bypass grafts) the illustrative embodiments will described and illustrated how segmented paths of the present invention may be used to accommodate naturally occurring vessels, such as, intercostals pairs 38, 40 and 42. Segmented cuff 1700 is secured in place around descending aorta 35 using fastening elements 1730. A single supply conduit 225 is used to port fluid into and out of the expandable layer (not shown) that is coupled to the cover layer 1720. The segmented cuff 1700 includes tab spacing profiles 1760, 1765 and 1770 to accommodate the intercostals pairs, respectively, 38, 40 and 42.

In contrast to the single supply conduit 225 utilized by the segmented cut 1700, the segmented cuff 1800 (FIG. 18A) has a single supply conduit 225 to supply fluid into a plurality of conduits, 1875, 1870, 1865, and 1860. The conduit 225 includes a first end configured to have a single lumen and a second end configured to have a plurality of lumens. In this illustrative embodiment, the plurality of lumens is four conduits 1875, 1870, 1865, and 1860. The conduits 1875, 1870, 1865, and 1860 are coupled via adapters 230 to the expandable layers (not shown) coupled to the inside of cover layer 1820. As with segmented cuff 1700, the segmented cuff 1800 also provides a plurality of tab spacing profiles to accommodate the intercostal pairs, 38, 40 and 42. The illustrated embodiment of the segmented paths 1800 also represents at least two different, expandable layer configurations. The segmented cuff 1800 may have four segmented layers each one being fed by an individual conduit. Thus, one expandable layer would be supplied by conduit 1860, another by conduit 1865, another by conduit 1870 and finally another supplied by conduit 1875. Alternatively, two or more expandable layers may be utilized in the segmented cuff 1800 where each of the two or more expandable layers is coupled to one or more of the conduits 1875, 1870, 1865, and 1860. For example, one expandable layer may be supplied by conduit 1875 and 1870 and another expandable layer may be supplied by conduits 1865 and 1860. In addition, embodiments of the expandable layer that may be used in the segmented cuff embodiments may be either segmented, continuous or combinations thereof. The embodiment of the segmented cuff 1800 also illustrates the configurability of the fluid supply to the segmented cuff 1800. It is to be appreciated that the diameter of the supply conduits connected to the cuff may be adjusted so that, for example, a larger diameter conduit is used to supply a larger volume, expandable layer. Consider, for example, where the segmented cuff 1800 has a single large, expandable layer supplied by conduit 1875 and three similarly sized but smaller, expandable layers supplied by the conduits 1870, 1865 and 1860. In this embodiment, the diameter of the conduit 1875 is larger than the diameters of conduits 1870, 1865, and 1860. As such, different diameter conduits may be used to advantage to provide more fluid as required by the sizes of the expandable layers being supplied. It is to be appreciated that the lumens in each of the conduits may, in an alternative embodiment, have the same diameter. Additionally, the diameter of the conduits and the impact on fluid supplied to the adjacent expandable layer may be used to improve synchronization of the activation of the cuff to a signal representing the cardiac cycle. In other words, supply conduit diameter may be useful when designing the overall fluid dynamics of an embodiment of a vascular assist system 100 of the present invention.

The segmented cuff embodiment 1850 of FIG. 18B provides a further advantage over the earlier described segmented cuff embodiments 1700 and 1800. The segmented cuff embodiment 1850 provides a plurality of individual cuffs 1830, 1840, 1850, and 1860 supplied by a single supply conduit 225. The sizes of the individual cuffs may be selected from a predetermined array of the sizes based on the average spacing of, for example, the intercostals pairs in a particular group of patients, such as, adult males. A different set of individual cuff sizes may be provided for an embodiment of a segmented cuff 1850 intended for use with a different group of patients with common physiology such as, for example, adult women, or children. While the segmented cuff embodiment 1800 has a desirable “single cuff” design, the individual cuffs of the segmented cuff embodiment 1850 provides an additional, different advantage. The individual cuffs of the segmented cuff 1850 may more readily adjusted to accommodate the particular vasculature found in a patient. Because each one of the cuffs may be individually placed and adjusted it is less likely that any one of the intercostal pairs would be damaged or impaired. As such, it is to be appreciated that the tab widths and tab spacing profiles of the various segmented cuff embodiments may be selected such that when the at least one tab on the first end is coupled to at least one tab on the second end when the cuff encircles a portion of a blood vessel. When in position to augment a vessel of interest, the segmented cuff embodiments if the present invention may be used to augment a vessel having a side branch (i.e., bypass graft) or branches (i.e., an intercostals pair) without restricting the flow of blood into or from the side branch. While the above embodiments have been described with reference to the descending aorta, it is to be appreciated that the segmented cuff embodiments may also be advantageously utilized on other vessels of interest, such as the vena cava or a portion of the aorta having a bypass graft.

It is to be appreciated that embodiments of the segmented cuffs described above may also be configured to provide a variety of augmentation schemes. An augmentation scheme refers to the order by which the cuffs in a segmented cuff or multiple cuff device are augmented. The cuffs may be configured to provide simultaneous compression or augmentation to the vessel of interest. Alternatively, the cuffs positioned along the length of a vessel may configured to provide sequential augmentation. Referring to FIG. 18B, sequential augmentation may be used when it is desired to have the cuffs activate in order, for example, in a direction proximate to the heart towards a direction distal from the heart. Using the segmented cuff 1850, for example, as illustrated on the descending aorta, the cuff 1830 would be activated first, followed by the cuff 1840, then the cuff 1850 and so forth. On the other hand, if the cuff 1850 where installed on, for example, the inferior vena cava then the augmentation scheme would be reversed (i.e., on the venous side augmentation would occur from a point distal to the heart moving towards the heart). Using this augmentation scheme, the cuff 1860 would be augmented first, followed by the cuff 1850, and then cuff 1840 and so forth. In either an arterial or a venous augmentation scheme, it is to be appreciated that in addition to the sequential augmentation described above, each of the cuffs may be activated simultaneously or nearly simultaneously to provide the desired augmentation.

Other variations in conduit design are possible in other additional embodiments. For example, the diameter of a single conduit may also be variable. As used herein, a variable diameter conduit refers to a conduit having a first diameter at a proximal location and a second, different diameter at a distal location. Consider the conduit 290 of FIG. 18C. The conduit 290 has a first diameter 291 proximate to the opening in the cover layer 220. The conduit 290 has a second, larger diameter 292 at a point distal from the cover layer 220. As such, conduit 290 represents an illustrative embodiment wherein a conduit 290 has a first diameter 291 adjacent to the opening 222 and a second different diameter 292 at a point distal to the opening 222. In an alternative embodiment, the larger diameter may be located proximate to the opening 222 and a smaller diameter may be used distal to the opening 222. The embodiments of the conduits used in the systems of the invention may have be a single uniform diameter conduit (conduit 225 in FIGS. 1 and 17); a single conduit coupled to the pump and plural conduits coupled to the device (FIGS. 18A and B); a mixture of larger diameter and smaller diameter conduits (FIGS. 18A and 18B) or have conduits having variable diameters (FIG. 18C).

Accordingly, there is provided a system having a plurality of cuffs, each of the plurality of cuffs including: (a) a compliant first layer configured to engage internal vasculature; (b) a second layer coupled to the first layer, the first layer and the second layer defining a cavity there between, the second layer defining an opening in communication with the cavity; and (c) a connector configured to couple the plurality of cuffs to one another. In one embodiment, the connector is coupled to the second layer of each of the plurality of cuffs and at least one of the plurality of cuffs is coupled to the vasculature of a body or, alternatively, to an organ in a body. In another embodiment, at least one of the plurality of cuffs is configured to engage with at least one set of intercostal arteries, at least one set of intercostal veins, the ascending aorta, the descending aorta, the superior vena cava, or the inferior vena cava.

In another embodiment, each of the plurality of cuffs is repeatably configurable between an uninstalled configuration and an installed configuration. In yet another embodiment, the second layer in each of the plurality of cuffs further comprises at least one pair of cooperative fastening elements. When a cuff is in the uninstalled configuration the at least one pair of cooperative fastening elements are uncoupled. When a cuff is in the installed configuration the at least one pair of cooperative fastening elements are coupled. In yet another embodiment, at least one pair of cooperative fastening elements has a plurality of fastening positions. The fastening position are arranged such that the size of the cuff (i.e, the diameter) in the installed configuration may be adjusted by changing to which of the plurality of fastening positions the other fastening element is coupled. In yet another embodiment, the second layer of each of the plurality of cuffs includes a first end and a second end. The first end and said second end are configured to be removeably coupled such that the cuff is reconfigurable between an uninstalled configuration in which the first and second ends are separate and an installed configuration in which the first and second ends are coupled.

In additional embodiments of a system having a plurality of cuffs there is also provided a pump in communication with the connector; and a controller for providing control signals to the pump in response to triggering signals from a cardiac cycle. In one embodiment, each of the plurality of cuffs is operated sequentially or simultaneously to provide vascular augmentation in a counterpulsation mode or a copulation mode.

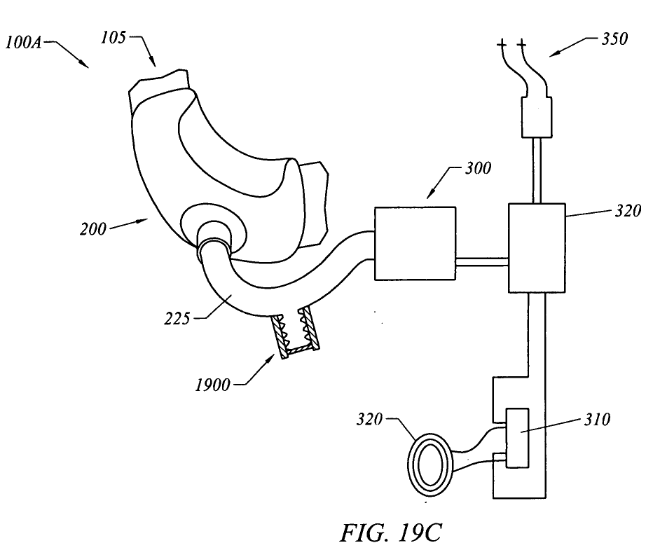

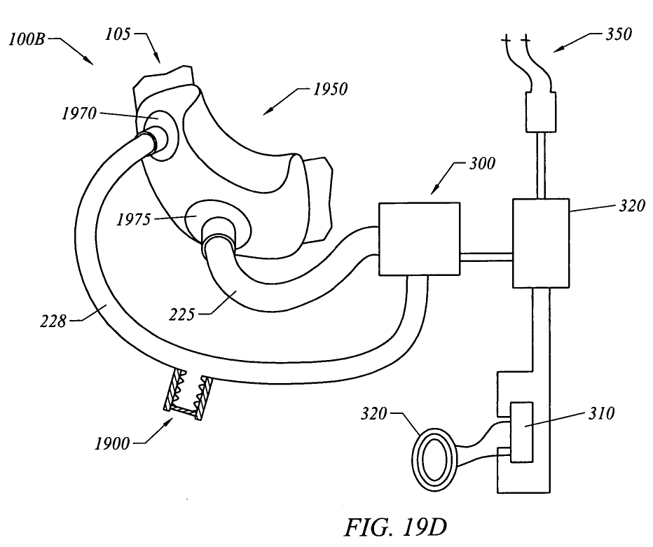

FIGS. 19A and 19B illustrate an embodiment of the fluid volume compensator 1900 that may be used with embodiments of the vascular assist system 100. The fluid volume compensator 1900 includes a housing, 1905 having a flexible bellows 1915. The flexible bellows 1915 has an interior volume that may be adjusted through movement of the adjusting means 1920. The interior of the flexible bellows 1915 is in fluid communication with the conduit 1910 that is or is in communication with the conduit 225 used to supply fluid to the embodiment of the cuff according to the present invention. The flexible bellows 1915 may be fabricated out of a material compatible with the fluids used in the vascular assist system 100. Suitable materials for the flexible bellows 1915 include, for example, silicone, neoprene and copolymers comprising styrene and butadiene.

Considering FIGS. 19A and 19B together, it will be appreciated that the flexible bellows 1915 as a variable volume that is configurable based on the position of the volume adjusting means 1920. FIG. 19A illustrates a low-volume displacement configuration, because the flexible bellows 1915 are positioned to store more fluid 1925. Because more fluid 1925 is present in the flexible bellows 1915, there is relatively less fluid 1925 available for the actuation (i.e., porting fluid into the cavity of the cuff) of the vascular assist cuff of the invention. As a result, the cuff augmentation force will be reduced. Alternatively, FIG. 19B illustrates a high volume displacement because the flexible bellows 1915 are configured to store less fluid 1925. Because less fluid 1925 is stored in flexible bellows 1915, there is relatively more fluid 1925 available for actuation of the cuff. As a result, the augmentation force of the cuff will be increased. Accordingly, the fluid volume compensator is configured to adjust the fluid volume ported into the cavity during activation of the cuff.

Volume adjusting means 1920 could be any structure or device used to configure the desired volume of the flexible bellows, and then secured that position. The adjusting means may be used, for example, when the vascular assist system is implanted to fine-tune the actuation force of the vascular assist device embodiment implanted in a body. In the illustrative embodiment, the volume adjusting means is a set screw. Alternatively, the fluid volume compensator 1900 may include a mechanism for adjusting the volume adjusting means 1920 in response to signals generated by the pump and pacing controller 320. For example, a servo controller or stepper motor could be suitably coupled to a volume adjusting means 1920, so that the volume adjusting means 1920 may be used to compress (FIG. 19B) or to expand (FIG. 19A) the flexible bellows 1915 under signals generated by the pump and pacing controller 320. Thus, embodiments of the fluid volume compensator 1900 under control of the pump and pacing controller 320 may adjust the volume of fluid available for actuation of the cuff. By adjusting the fluid volume available for the cuff one may very the amount of augmentation provided by the cuff or vascular assist device of the invention. Additionally, the fluid volume compensator 1900 may act to allow replenishment of the fluid in the vascular assist system 100. When functioning as allow replenishment of fluid (i.e., as a reservoir), the flexible bellows 1915 are initially in an expanded position (FIG. 19A). As fluid loss occurs in the system or if system performance degrades (i.e., low augmentation pressure) in a matter suggesting system volume has decreased, then the volume adjusting means 1920 can be moved to compress the flexible bellows 1915 into a configuration where less fluid is stored in the flexible bellows 1915 (i.e., as in FIG. 19B) and relatively more fluid is available for use in the system to activate the cuff.

FIGS. 19C and 19D illustrate an embodiment of the fluid volume compensator 1900 installed in alternative embodiments of the vascular assist system 100. In FIG. 19C, the vascular assist system 100A includes a fluid volume compensator 1900 is installed in the conduit 225 between the pump 300 and the vascular assist device 200. In FIG. 19D, the vascular assist system 100B illustrates a multiple conduit embodiment where multiple conduits (i.e., two in this embodiment, more are possible in other configurations) are used to supply fluid into the cuff 1950 and return fluid to the pump 300. In the illustrated embodiment, one conduit 225 is used to supply fluid into the a cuff 1950 via coupling 1975 near the center of the cuff 1950. A second conduit 228 is coupled to the distal end of cuff 1950 via coupling 1970 to return fluid to the pump 300. It is to be appreciated that the cuff 1950 have openings 222 adjacent the couplings 1970 and 1975 that provide access into the cavity 250 (not shown) within the cuff 1950. In this embodiment, the fluid volume compensator 1900 is installed in the return conduit 228. In this embodiment, the supply conduit 225 has a larger diameter than the return conduit 228. It is to be appreciated that in other embodiments, the fluid volume compensator 1900 may be installed on the fluid supply conduit 225 instead of the fluid return conduit 228. In the embodiments where the fluid volume compensator is disposed between the pump 300 and cuff 200 the fluid volume compensator is disposed in a fluid flow path between the pump and the cavity.

In the illustrated embodiment, the supply conduit 225 and coupling 1975 have a larger diameter than the return conduit 228 and coupling 1970. In alternative embodiments, the supply conduit may be coupled to the proximate end of the cuff 1950 while the return conduit is coupled to the distal end of the cuff 1950 and vice versa. In another embodiment, the return conduit may be centrally coupled to the cuff 1950 and supply conduit may be have plural conduits coupled to, for example, the proximal and distal ends of the cuff 1950. In yet another alternative embodiment, a single supply conduit may be coupled to the central portion of the cuff 1950 and plural return conduits may be coupled to the proximal and distal ends of the cuff 1950. return may be conduit and coupling 228, 1970 may have a larger diameter than the supply conduit and coupling 225, 1975. Accordingly, embodiments of the cuff 1950 may include plural openings 222 having a variety of diameters and locations about the cuff 1950. In addition, it is to be appreciated that the various diameter conduits (FIGS. 18A and 18B) or variable diameter conduits (FIG. 18C) may also be utilized in the multiple conduit embodiments described above. Each of the above described conduit, opening and coupling configurations described above may be used to advantage to control the fluid flow within vascular assist system embodiments of the present invention.

Turning now to FIGS. 20A through 30 various alternative fastener embodiments will be described. As described above with regard to FIGS. 2-4, fastening means 280 is provided to secure the ends of the cover layer about the vessel of interest. When the cover layer includes a first end and a second end, the first end and the second end are configured to be removeably coupled. Thus, the vascular assist device is reconfigurable between an uninstalled configuration in which the first and second ends are separate and an installed configuration in which the first and second ends are coupled. The various anchoring, fastening, or connection mechanisms described below may be used for disposing embodiments of the cuffs of the present invention around the vasculature to be augmented. It is to be appreciated that each of the fastening means described herein allow the cuff embodiment to be moved into and out of its second or operational configuration with ease. Each of the fastening means and securing means embodiments below can be readily adjusted, repositioned and/or removed as will be described further in the discussion that follows.

The various fastening element embodiment have a number of features in common. With the exception of cuff embodiments using sewed or sutured ends (FIGS. 9 and 12), the cover layer of each cuff includes at least one pair of cooperative fastening elements. The fastening element embodiments may are repeatably configurable between an uninstalled configuration and an installed configuration. When the vascular assist device or cuff embodiment is in the uninstalled configuration, the at least one pair of cooperative fastening elements are uncoupled. When the vascular assist device or cuff embodiment is in the installed configuration, the at least one pair of cooperative fastening elements are coupled. As earlier described with regard to FIGS. 2 and 3, one of the fastening elements in the at least one pair of cooperative fastening elements includes a plurality of fastening positions. The plurality of fastening positions are configured such that the size of the device in the installed configuration may be adjusted by changing to which of the plurality of fastening positions the other fastening element is coupled.

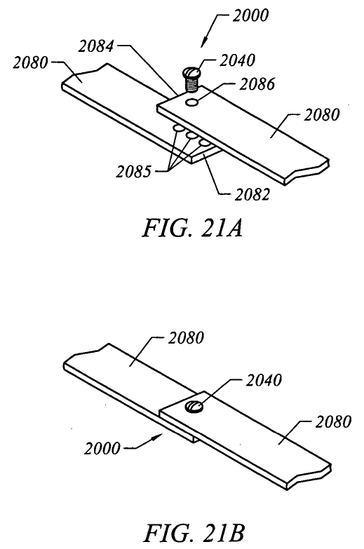

FIGS. 20A through 21b illustrate a fastener embodiment 2000 using a screw 2040 and screw receiving plate 2084 having plural positions 2085. The fastener embodiment 2000 may be attached to the flaps 270. The ends of the fastening elements 2082, 2084 are placed into an overlapping position (i.e., ends 2082 and 2084 overlap) when the cuff is installed about a vessel (not shown) (FIG. 21A). As the end 2084 (i.e., end with the fastening plate 2087) is moved between the fastening positions 2085 on the end 2082, the size of the cuff is adjusted. When the hole 2086 is positioned above the desired receiving hole 2085, a fastener 2040 is placed through the hole 2086 and fastened to the plate 2084. The hole 2086 in the plate 2087, fastener 2040 and receiving holes 2085 are all similarly sized and threaded to operate together to secure an embodiment of the cuff about a vessel.

In the illustrated embodiment, the plate 2084 and 2087 may be metal plates integrally formed within or between layers of the fastening elements 2080. The metal strips 2084, 2087 may be stainless steel or other suitable materials such as titanium, titanium alloys, nylon, ABS, etc. The strips can be inserted in the flaps 227 during or after fabrication of the second layer 220. To improve adhesion of the metal strips 510, 520 to the flaps 227 of the second layer 220, the stainless steel strips 510, 520 can be coated with a primer.

In use, when the cuff 200 is positioned around the vessel, the appropriate opening 2085 is selected based on the size (i.e., circumference) of the vessel of interest (i.e., the aorta). A screw 2040 is inserted into the opening 2086 and threaded into the selected opening 2085. The fastener 2000 can be readily adjusted and/or removed by removing the screw 2040 and removing or repositioning the device 200. The screw 2040 is dimensioned such that it securely engages the threaded opening 2085, but does not extend past the cover layer. In other words, the screw 2040 does not compress the vessel.

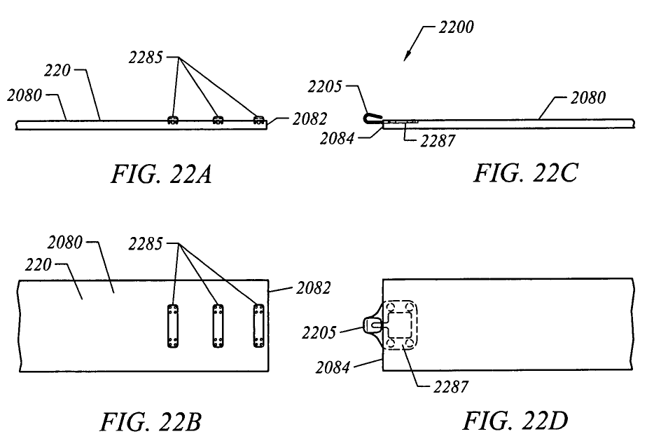

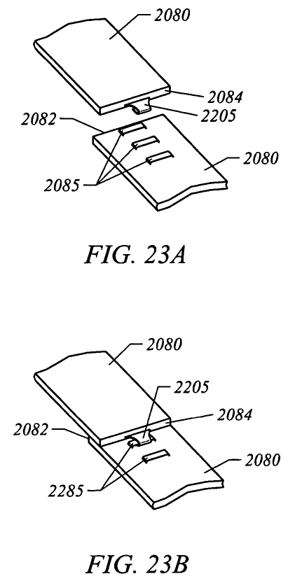

FIGS. 22A-22D and 23A and 23B are hook 2205 and anchor bars 2285 fasteners that illustrate an embodiment of a connection mechanism 2200 that can be disposed on opposing flaps 270 described above. The connection mechanism 2200 includes at least one anchor bar 2285 in one end 2082 of the opposing flap 270. In the illustrated embodiment, three anchor bars 2285 are illustrated. The anchor bar 2285 is a raised strip that is coupled to the second layer 220 at two ends and defines a clearance between the anchor bar 2285 and the second layer 220. The other flap 227 includes a metal strip 2287 with a buckle 2084 defined thereon on the other end 2084. The anchor bar 2285 and the buckle 2205 may be stainless steel or other suitable materials such as titanium, titanium alloys, nylon, ABS, etc. The anchor bar 2285 and the buckle 2205 can be inserted in the flaps 227 during or after fabrication of the second layer 220. To improve adhesion of the anchor bar 2285 and the buckle 2205 to the flaps 227 of the second layer 220, the anchor bar 2285 and the buckle 2205 can be coated with a primer.

In use, when the cuff 200 is positioned around the aorta, the appropriate anchor bar 2285 is selected based on the size (i.e., circumference) of the vessel. The buckle 2205 is positioned to engage the selected anchor bar 2285 through the clearance defined between the anchor bar 2285 and the second layer 220. The connection mechanism 2200 can be readily adjusted and/or removed by disengaging the buckle 2205 from the anchor bar 2285 and removing or repositioning the device 200.

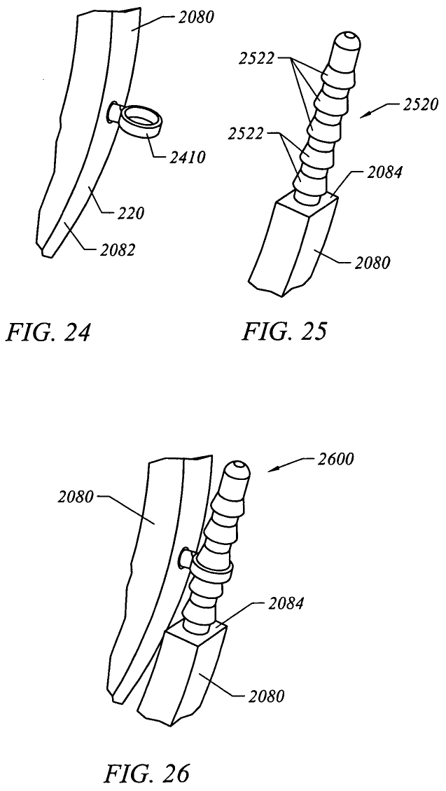

FIGS. 24, 25 and 26 illustrate an embodiment of a lock-tie wrap fastener 2600 components of the lock-tie wrap fastener 2600 can be disposed on opposing flaps 270 described above. The connection mechanism 2600 includes a locking ring 2410 on one of the opposing flaps having end 2082. The locking ring 2410 is a raised ring that has one end embedded in the second layer 220 of the cuff 200. The other flap 227 includes a mating element 2520 that is has multiple identical locking portions 2522. Each locking portion 2522 is configured to be pushed through the locking ring 2410, but is unable to be pulled back through the locking ring 2410. In this manner, one end 2084 with the mating element 2520 can be pushed through the other end 2082 having locking ring 240 until a secure fit is achieved. The locking ring 2410 and mating element 2520 may be stainless steel or other suitable materials such as titanium, titanium alloys, nylon, ABS, etc. The locking ring 2410 and the mating element 2520 can be inserted in the flaps 270 during or after fabrication of the second layer 220. To improve adhesion of the locking ring 2410 and the mating element 2520 to the flaps 270 of the second layer 220, the locking ring 2410 and the mating element 2520 can be coated with a primer. There is provided a cuff securing device wherein the mating fasteners include positive-locks. While the illustrative embodiment uses generally circular positive lock features, it is to be appreciated that other positive lock features are possible. The positive lock feature is the feature that holds the mating pieces in place and could have virtually any shape such as, for example, ring, square or other shape so long as holds the mating pieces into a unidirectionally oriented relationship.

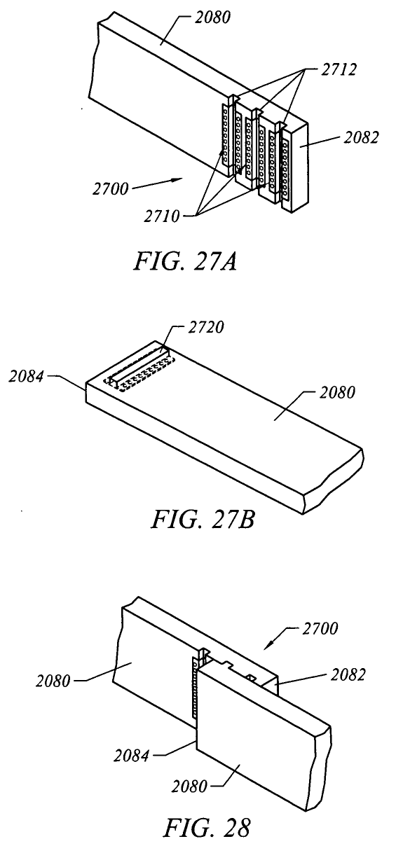

FIGS. 27A, 27B and 28 illustrate an embodiment of a connection mechanism 2700 that can be disposed on opposing flaps 227 described above. The connection mechanism 2700 includes embedded magnetic material 2710 in one of the opposing flaps. The other flap 270 includes an embedded magnet 2720. The magnetic material 2710 and the magnet 2720 can be inserted in the flaps 270 during or after fabrication of the second layer 220. To improve adhesion of the magnetic material 2710 and the magnet 2720 to the flaps 270 of the second layer 220, the magnetic material and the magnet may be coated with a primer.

In the illustrated embodiment, the magnetic material 2710 is disposed about channels or grooves 2712 defined along the flap 2080. Moreover, the magnet 2720 is disposed externally to the opposing flap adjacent end 2084. In this manner, the magnet can engage the groove 2712 to achieve a secure coupling in which there is a greater interface between the magnetic material 2710 and the magnet 2720.

In use, when the cuff 200 is positioned around a vessel, the magnet 2720 is aligned with the appropriate groove 2712 based on the size (i.e., circumference) of the vessel. The magnet 2720 is positioned to engage the selected groove 2712 and the corresponding embedded magnetic material. The magnetic connector 2700 can be readily adjusted and/or removed by disengaging the magnet 2720 from the groove 2720 and removing or repositioning the device 200. Accordingly, there are embodiments of the magnetic coupler system 2700 where the cover layer 220 includes at least one pair of cooperative magnetic fastening elements. In a representative embodiments, at least one of the mating fasteners is magnetic. In another representative embodiment, there is provided a magnetic coupling system where one of the cooperative mating fasteners is a magnet and the other mating fastener is formed from a magnetically attractive material.

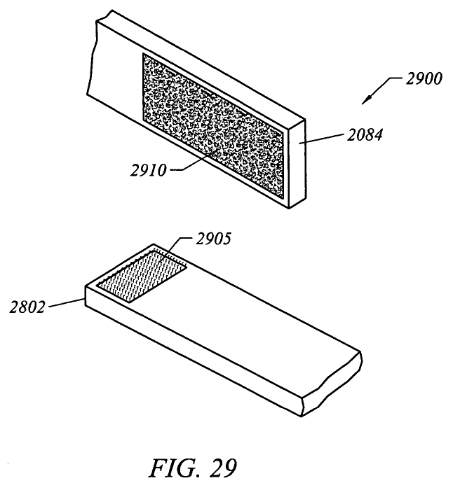

FIG. 29 illustrates an embodiment of a fastening system 2900 for use with cuff embodiments of the present invention. One flap 270 with end 2082 includes plural fastening hooks 2905. The flap 270 having the other end 2084 includes plural eyes or loops 2910 configured to engage with the plural hooks 2905. The plural hooks 2095 and plural loops 2910 may be, for example, strips of suitably sized Velcro™. The hook and loop material may be inserted into the flaps 227 during or after fabrication of the second layer 220. To improve adhesion of the hook and loop material to the second layer 220, the hook and loop material may be coated with a primer or other suitable adhesive.

In use, when the cuff 200 is positioned around a vessel, a portion of the plural hooks 2095 is aligned with the appropriate portion of the plural loops 2910 based on the size (i.e., circumference) of the vessel. The plural hooks 2095 are positioned to engage the selected portion of the plural loops 2910. The fastening system 2900 can be readily adjusted and/or removed by disengaging the plural hooks 2095 from the portion of the plural loops 2910. Thus, there is provided an embodiment of a fastener having mating fasteners that include a hook and a loop. In an alternative embodiment, the there is provided an embodiment of a fastener having mating fasteners that include a plurality of hooks and a plurality of loops.

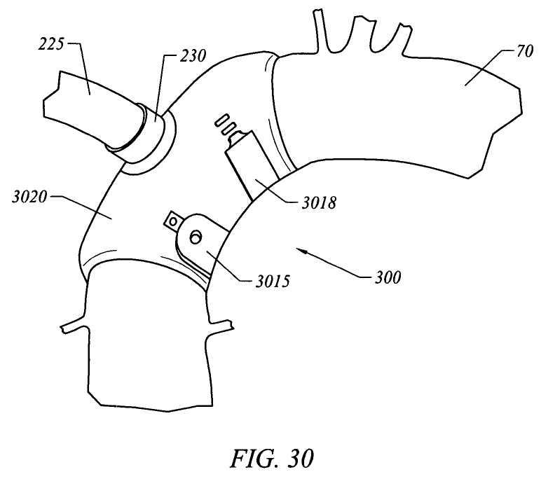

A number of different fastener embodiments have been described. It is to be appreciated that cuff embodiments of the present invention may employ a single fastening system or multiple fastening systems to be secured about a vessel. In addition, the multiple fastening systems are not limited to only including fastening elements of only one type. Consider the cuff embodiment illustrated in FIG. 30. The illustrated cuff 3000 is secured about the vessel 70 using two different fastening systems 3015, 3018. Fastening system 3015 may be, for example, similar to fastener 280 illustrated in FIG. 7. Fastening system 3018 may be, for example, the hook and anchor system illustrated in FIG. 23B. In addition, the fastening systems of the present invention are limited to the generally orthogonal orientation relative to the cover layer 220 as illustrated in FIGS. 2, 3 and 4. Fastening systems 3015 and 3018 also represent how the fastening systems may be configured in an angular arrangement on the cover layer 220. In the illustrative embodiment of FIG. 30, the angular arrangement of the fastening systems 3015 and 3018 is selected to further conform the cover layer 3020 about the curves vessel 70. Accordingly, the fastening system embodiments of the present invention may include a mixture of securing systems and angular orientations to ensure greater compliance when secured about a vessel of interest. In the illustrative embodiment, the angled position of the fastening systems 3015, 3018 may be adjusted to accommodate other vessel curvature as well.

In some embodiments of the vascular assist system 100, the components of the system in contact with the vessel to be augmented are coated with a material to compounds to enhance tissue growth and prevent fibrosis. As is conventional with implants in a body, scar tissue begins forming around the device as the natural healing response of the patient body. This scar tissue is fibrous in nature and non-elastic. The scar tissue becomes eventually becomes very hard and compromises the compliance of the vessel being augmented. In order to avoid this undesired reaction in the patient body, embodiments of the cuff are coated with a tissue growth inducing polymeric material. The tissue growth inducing polymeric material promotes the growth of healthy tissue growth in place of scar tissue. Consider an embodiment where the outer surface (i.e., all surfaces in contact with the patient body) are coated with a healthy tissue growth material. This cuff embodiment may be implanted about a vessel, for example, using a cuff that may be sutured in place about the vessel (i.e., cuffs 390 and 405). In this embodiment, the coated cuff is sutured in place using absorbable sutures. Absorble sutures are absorbed into the body after a period of time, usually less than one week. The sutures are selected to remain for sufficient duration such that healthy tissue growth occurs on the coated cuff embodiment. As healthy tissue growth occurs, the cuff will become attached to the vessel via the healthy tissue. Through the use of the tissue growth material coating on embodiments of the cuff a more compliant and less damaging cuff fastening means is provided. As such, it is possible to coat the cuff with a tissue growth inducing polymeric material to enhance tissue growth and prevent fibrosis. For example, many proteins are known to promote healthy tissue growth. Additionally, there exist protein and polymer materials that may be used to enhance tissue growth and prevent fibrosis. Examples of tissue growth inducing polymeric materials that may be used with embodiments of the present invention include poly-L-lysine and poly-D-lysine. While described above with regard to using sutures as the cuff securing means, it is to be appreciated that embodiments of the present invention may employ other cuff fastening means described herein in conjunction with the tissue growth inducing polymeric materials discussed herein. In the embodiments where additional fastening elements are used, the fastening elements would also be coated with the tissue growth inducing polymeric material.

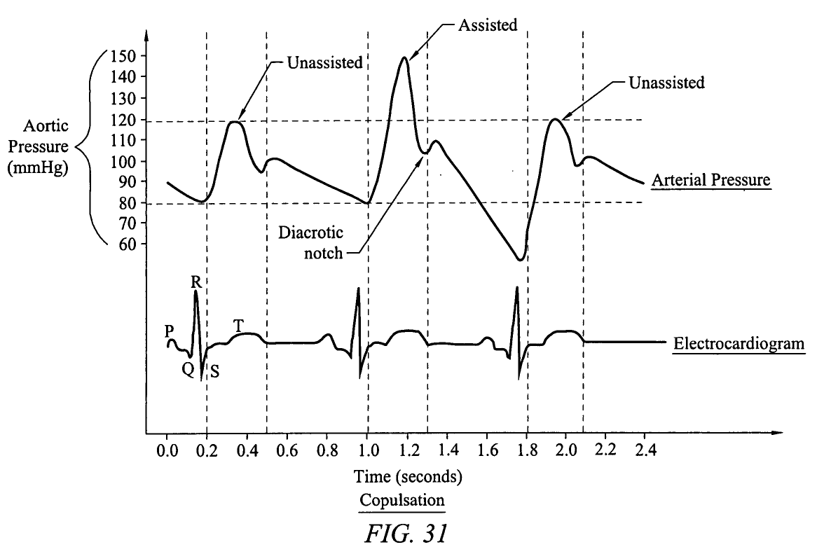

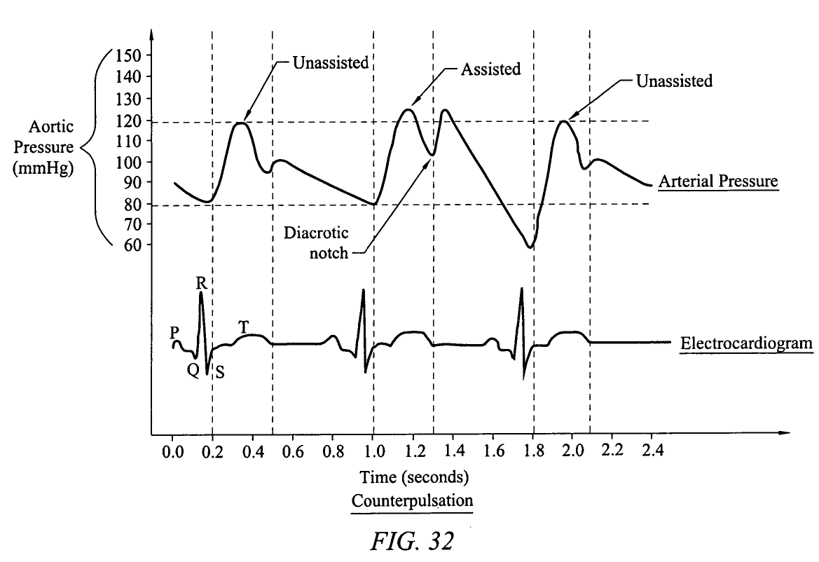

Referring now to FIGS. 31 and 32, exemplary electrocardiogram (ECG) readouts are illustrated. FIG. 31 illustrates a comparison of arterial pressure and a corresponding ECG readout when an embodiment of the vascular assist system 100 is providing augmentation is a copulsation pattern. FIG. 32 illustrates a comparison of arterial pressure and a corresponding EKG readout when an embodiment of the vascular assist system 100 is providing augmentation is in a counterpulsation manner.

In FIG. 31 the ECG is processed by the pacing and pump controller 320 and an R-wave is detected. Next, the pacing and pump controller 320 determines the heart rate using the R-R intervals. In order to inflate the cuff to provide copulsation, the pacing and pump controller 320 triggers the pump at about 90% rise of the R-wave. Depending on the desired dwell-time (i.e., length of time the cuff is inflated) the signal ON duration can be programmed. In this augmentation pattern, the pump shuttles the fluid from the reservoir to the cuff and inflates the cuff during the ventricular systole. In this matter, the cuff helps the heart by pushing the blood at a higher pressure. An additional benefit of this augmentation pattern is that it makes the blood flow away from the aorta faster into the side branches. When the desired dwell time (i.e., duration that cuff is inflated) has elapsed, the pacing and pump controller 320 signals for the pump to shuttle fluid back from the cuff into the reservoir (i.e., the cuff deflates). As the cuff deflates, the augmented vessel wall also relaxes. This action reduces the pressure in the aorta thus reducing the work load for the heart for the following beat.

FIG. 31 illustrates 1:2 augmentation. 1:2 augmentation means that there is one assisted heartbeat for every two unassisted heartbeats. There are three heart beats shown. First and the third heart beats (t=0.2 and t=1.8) are un-assisted and the second heart beat (t=1.0) is assisted. End-systolic pressure of the assisted beat (i.e., about 125 mm Hg) is higher compared to that of an unassisted beat (i.e., about 120 mm Hg). This increase in end-systolic pressure is known as systolic augmentation. Systolic augmentation is desired because it helps the blood flow faster at a higher pressure. The end-diastolic pressure in the second assisted beat (t=1.8, about 60 mm Hg) is lower that of an unassisted beat (t=1.0, about 80 mm Hg). This reduction in end-diastolic pressure is known as after-load reduction. As a result of after load reduction, there is less pressure in the aorta and the heart does not have to work as hard to pump the blood for the following beat. After load reduction thus reduces the workload of the heart. While the above embodiments are described using triggering based on the ECG readings, it is to be appreciated that augmentation in a co-pulsation pattern may also be triggered based on blood pressure, either venous pressure or arterial pressure.

As with FIG. 31, the ECG in FIG. 32 is processed by the pump and pacing controller 320 and an R-wave is detected. Next, the pump and pacing controller 320 determines the heart rate using the R-R intervals. In order to inflate the cuff to provide counterpulsation the pump and pacing controller 320 calculates the Q-T interval for the heart rate and triggers the pump at the end of the T-wave. Depending on the desired dwell-time the signal ON duration can be programmed. The pump shuttles the fluid from the reservoir to the cuff and inflates the cuff during the ventricular diastole. This increases the blood flow into the coronaries and other side branch arteries. When the pump shuttles the fluid back from the cuff into the reservoir the cuff deflates. This action reduces the pressure in the aorta thus reducing the work load for the heart for the following beat.

FIG. 32 shows 1:2 augmentation. There are three heart beats shown. First and the third heart beats are un-assisted (t=0.3 and t=2.0) and the second heart beat is assisted (t=1.2). Peak pressure after the diacrotic notch in the assisted beat (t=1.4, about 125 mm Hg) is greater than the peak pressure of an unassisted beat (t=0.5, less than about 100 mm Hg). This increase in secondary peak pressure provides the desired diastolic augmentation. Diastolic augmentation is desired because it increases the blood flow into the coronaries and other arteries. The end-diastolic pressure in the second assisted beat (t=1.8, about 60 mm Hg) is lower that of an unassisted beat (t=1, about 80 mm Hg). This reduction in end-diastolic pressure provides the benefits of after-load reduction as discussed above. While the above embodiments are described using triggering based on the ECG readings, it is to be appreciated that augmentation in a counter pulsation augmentation pattern may also be triggered based on blood pressure, either venous pressure or arterial pressure.

In addition, the R-R interval is calculated by a using a rolling average of R-waves based on real time heart rate changes. As the heart rates changes, so then changes the R-R interval. The pump and pacing controller 320 has software programs and electronics to record and average the R-R interval and adjust the system and cuff as needed. It is to be appreciated therefore that the augmentation patterns provided above may also advantageously utilize the rolling R-R wave averages.

As discussed above, the cuff embodiments and the vascular augmentation system embodiments above may be used to in a method for augmenting blood flow in a patient body. First, detect a first cardiac cycle trigger. Next, port fluid into the cavity of the cuff so as to elastically deform the first layer thereby compressing a blood vessel in response to the first cardiac cycle trigger. Then, port the fluid out of the cavity in response to a second cardiac cycle trigger. The first cardiac is related to an ECG of the patient. Alternatively, the first cardiac trigger is related to the increasing portion of the R-wave. In another alternative embodiment, the first cardiac trigger occurs at 90% of the increasing R-wave amplitude. In another embodiment, the first cardiac trigger is related to the ECG of the patient and selected so that the step of porting a fluid into the cavity so as to elastically deform the first layer coincides with the ventricular systole. In yet another embodiment, the first cardiac trigger is related to the Q-T interval, to the decreasing portion of the T-wave or the end of the T-wave. In yet another embodiment, the first cardiac trigger is related to the T-wave and selected so that the step of porting a fluid into the cavity so as to elastically deform the first layer coincides with the ventricular diastole.

In yet another embodiment, the second cardiac cycle trigger is a predetermined time limit. In yet another embodiment, the second cardiac cycle trigger is based on the R-R interval. There is also provided an additional embodiment where the second cardiac cycle trigger is related to aortic pressure, a predetermined time limit, or is based on the R-R interval. In another embodiment, the first and the second cardiac cycle triggers are selected to operate the cuff in compulsation mode. In another embodiment, the cavity inflates during the ventricular systole of the heart. In yet another embodiment, the first and the second cardiac cycle triggers are selected to operate the cuff in counterpulsation mode.

There is also provided another method for augmenting blood flow in a body where a cardiac cycle trigger is detected. Fluid is ported into a cavity so as to elastically deform the first layer in response to the cardiac cycle trigger. The vessel is held compressed for a known duration and then fluid is ported out of the cavity in order to allow the vessel to relax. This method may utilize the cardiac trigger and augmentation modes described above.

In an alternative embodiment, the method may be performed in a coplusation manner wherein the cardiac trigger is related to the aortic pressure and selected so that the step of porting a fluid into the cavity so as to elastically deform the first layer coincides with the ventricular systole. Alternatively, the method may be performed in a counterpulsation manner, wherein the cardiac trigger is related to detecting R-wave of the ECG, computing the Q-T interval and triggering the pump to coincide with the end of the T-wave for porting the fluid into the cavity so as to elastically deform the first layer and compress the blood vessel. In yet another alternative, the method may be performed in a counterpulsation manner, wherein the cardiac trigger is related to detecting the peak aortic pressure and computing the duration for the aortic valve to close and triggering the pump for porting the fluid into the cavity so as to elastically deform the first layer and compress the blood vessel to coincide with the aortic valve closing.

In yet another alternative embodiment, there is provided a method for augmenting blood flow in a vessel of a patient that includes changing the pressure of a fluid in the cavity based on a signal associated with the cardiac cycle; deforming the first layer in response to the changing pressure of the fluid in the cavity; and deforming the walls of a vessel at least partially encircled by the first layer in response to the deforming of the first layer. This method may also utilize any of the above mentioned trigger and timing sequences described above. In addition, there is provided an embodiment where the method includes a signal associated with the cardiac cycle is related to the ECG of the patient and selected so that the step of deforming the walls of a vessel at least partially encircled by the first layer in response to the deforming of the first layer coincides with the ventricular systole. Alternatively, the changing the pressure of a fluid in the cavity is occurring so that the pressure in the cavity is increasing during the ventricular systole of the heart. Alternatively, the signal associated with the cardiac cycle is related to the T-wave and selected so that the step of changing the pressure of a fluid in the cavity coincides with the ventricular diastole. Embodiments of the present method may be operated in either or both of co-pulsation or counter pulsation mode.

### CONCLUSION

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalence.

The previous description of the preferred embodiments is provided to enable any person skilled in the art to make or use the present invention. While the invention has been particularly shown and described with reference to preferred embodiments thereof, it will be understood by those skilled in art that various changes in form and details may be made therein without departing from the spirit and scope of the invention.

1. A device, comprising:
a compliant first layer configured to engage internal vasculature;
a second layer coupled to the first layer, the second layer having a stiffness greater than a stiffness of the first layer; and
the first layer and the second layer defining a cavity therebetween, the cavity having a volume wherein the first layer is configured to be deformed in response to a change in the volume of the cavity.

2. The device of claim 1 wherein the first layer is selectively radially deformed in response to a change in the volume of the cavity.

3. The device of claim 1, wherein the first layer is fabricated with a first material and the second layer is fabricated with a second material.

4. The device of claim 3, wherein the first material is a first silicone elastomer and the second material is a second silicone elastomer.

5. The device according to claim 1 wherein a portion of the device is coated with a tissue growth inducing polymeric material.

6. The device according to claim 5 wherein the tissue growth inducing material is one of poly-L-lysine and poly-D-lysine.

7. The device of claim 4, wherein the first silicone elastomer is a 5-50 A silicone elastomer having a minimum of 500% elongation.

8. The device of claim 4, wherein the second silicone elastomer is a 65-95 A silicone elastomer having less than a 400% elongation.

9. The device of claim 1 wherein the second layer further comprises a reinforcement element coupled to the second layer such that the reinforcement element is configured to maintain the length and width of the second layer.

10. The device of claim 4, wherein the first material is an elastomer that has hardness of 5-50 shore A and having a minimum elongation of 500%.

11. The device of claim 4, wherein the second material is an elastomer that has hardness of 65-95 shore A and having a maximum elongation of 400%.

12. The device of claim 9, wherein the reinforcement element is fabricated from at least one of polyester, nylon, para-aramid fiber, stainless steel, platinum, superelastic nitinol and alloys of nickel and titanium.

13. The device of claim 3, wherein the first and second material are one of silicone, neoprene and copolymers comprising styrene and butadiene.

14. The device of claim 1, wherein the first layer is coupled to the second layer about a perimeter of the first layer.

15. The device of claim 1, wherein the first layer is coupled to the second layer about a portion of the perimeter of the second layer.

16. The device of claim 14, wherein a perimeter of the second layer extends beyond the perimeter of the first layer.

17. The device of claim 1, the second layer further comprising a length and a width, the first layer further comprising a length and a width, wherein the length of the first layer is less than the length of the second layer.

18. The device of claim 17, wherein the width of the first layer is less than the width of the second layer.

19. The device of claim 17 wherein the length of the second layer is sufficient for the second layer to completely encircle a portion of a blood vessel.

20. The device of claim 19 having a second layer further comprising a fist end and a second end wherein when the second layer is configured to completely encircle a portion of a blood vessel, the first end and the second end of the second layer overlap.

21. The device of claim 19 wherein the portion of the blood vessel comprises the ascending aorta.

22. The device of claim 19 wherein the portion of the blood vessel comprises the descending aorta.

23. The device of claim 19 wherein the portion of the blood vessel comprises a set of intercostal arteries or a set of intercostal veins.

24. The device of claim 19, wherein the portion of the blood vessel comprises the superior vena cava.

25. The device of claim 19, wherein the portion of the blood vessel comprises the inferior vena cava.

26. The device of claim 1 wherein the second layer further comprising a first end and a second end, wherein each of the first end and the second end have at least two tabs, each of the tabs in the at least two tabs has a width wherein the sum of the widths of all the tabs in the at least two tabs on the first end is less than the width of the device.

27. The device of claim 26, wherein the at least two tabs on the first and second ends are configured to be removably coupled such that the device is reconfigurable between a first configuration in which the at least two tabs on the first and second ends are separate and a second configuration in which the at least two tabs on the first and second ends are coupled.

28. The device of claim 26 wherein a tab spacing profile is provided between adjacent tabs in the at least two tabs, the tab spacing profile having a width wherein the sum of the tab spacing profile widths and the widths of all of the tabs in the at least two tabs equals the width of the device.

29. The device of claim 28 wherein the tab spacing profile between each of the tabs in the at least two tabs is the same.

30. The device of claim 28 having at least two different tab spacing profiles.

31. The device of claim 26 wherein when the device is disposed about a blood vessel in a body at least one of the two or more tabs of the first end is coupled to at least one of the two or more tabs on the second end.

32. The device of claim 28, wherein the tab widths and tab spacing profiles are selected such that when the at least one tab on the first end is coupled to at least one tab on the second end the device encircles a portion of a blood vessel and a side branch coupled to the blood vessel without restricting blood flow into or from the side branch.

33. The device of claim 32, wherein the portion of a blood vessel and a side branch comprises the descending aorta and at least one set of intercostal arteries.

34. The device of claim 32, wherein the portion of a blood vessel and a side branch comprises the inferior vena cava and at least one set of intercostal veins.

35. The device of claim 20, wherein the second layer first and second ends are configured to be removably coupled such that the device is reconfigurable between a first configuration in which the first and second ends are separate and a second configuration in which the first and second ends are coupled.

36. The device of claim 1 wherein the perimeter of the first layer perimeter defines a first shape and the perimeter of the second layer defines a second shape.

37. The device of claim 36 wherein the first shape is similar to the second shape.

38. The device of claim 36 wherein the second shape is rectangular and first shape is a different shape than the second shape.

39 The device of claim 1 further comprising an expandable bladder having a volume and disposed within the cavity and configured such that the first layer is deformed in response to a change in volume of the bladder.

40 device, comprising:
an expandable layer configured to engage internal vasculature;
a cover layer coupled to the expandable layer and having a length and a width, the expandable layer and the cover layer defining a cavity therebetween, the cavity having a volume, the cover layer defining an opening in fluid communication with the cavity; and
a reinforcement element coupled to the cover layer and configured to maintain the length and width of the cover layer, wherein, the expandable layer is configured to be selectively deformed in response to a change in the volume of the cavity.

41. The device of claim 40, wherein selectively deformed is a deformation that is radially selective.

42. The device of claim 40, selectively deformed is a deformation that is longitudinally selective.

43. The device of claim 40, wherein the first layer is fabricated with a first material having a first stiffness and the second layer is fabricated with a second material having a second stiffness.

44. The device of claim 43 wherein the second stiffness is greater than the first stiffness.

45. The device of claim 44, wherein the first material is a first silicone elastomer and the second material is a second silicone elastomer.

46. The device of claim 45, wherein the first silicone elastomer is a 5-50 A silicone elastomer having a minimum of 500% elongation.

47. The device of claim 45, wherein the second silicone elastomer is a 65-95 A silicone elastomer having less than a 400% elongation.

48. The device of claim 40, wherein the reinforcement element is at least one of polyester, nylon, para-aramid fiber, stainless steel, platinum, superelastic nitinol and alloys of nickel and titanium.

49. The device of claim 45, wherein the first material is an elastomer that has hardness of 5-50 shore A and having a minimum elongation of 500%.

50. The device of claim 45, wherein the second material is an elastomer that has hardness of 65-95 shore A and having a maximum elongation of 400%.

51. The device of claim 43, wherein the first material and second material are one of silicone, neoprene and copolymers comprising styrene and butadiene.

52. The device of claim 40, wherein the device is reconfigurable between a first, substantially planar configuration and a second, substantially tubular configuration.

53. The device of claim 52, further comprising a connection member joining a first portion of the cover layer to a second portion of the cover layer when the device is in the second configuration.

54. The device of claim 40, wherein the cover layer includes a first end and a second end, said first end and said second end configured to be removably coupled such that the device is reconfigurable between a first configuration in which the first and second ends are separate and a second configuration in which the first and second ends are coupled.

55. The device of claim 54 wherein when the first end and the second end are coupled the first end and the second end are in an overlapping configuration that encircles a portion of a blood vessel.

56. The device of claim 55 wherein the portion of the blood vessel comprises the ascending aorta.

57. The device of claim 55 wherein the portion of the blood vessel comprises the descending aorta.

58. The device of claim 55 wherein the portion of the blood vessel comprises a set of intercostal arteries or a set of intercostal veins.

59. The device of claim 55, wherein the portion of the blood vessel comprises the superior vena cava.

60. The device of claim 55, wherein the portion of the blood vessel comprises the inferior vena cava.

61. The device of claim 40 wherein the cover layer further comprises a first end and a second end, wherein each of the first end and the second end have at least two tabs, each of the tabs in the at least two tabs has a width wherein the sum of the widths of all the tabs in the at least two tabs on the first end is less than the width of the device.

62. The device of claim 61, wherein the at least two tabs on the first and second ends are configured to be removably coupled such that the device is reconfigurable between a first configuration in which the at least two tabs on the first and second ends are separate and a second configuration in which the at least two tabs on the first and second ends are coupled.

63. The device of claim 61 wherein a tab spacing profile is provided between adjacent tabs in the at least two tabs, the tab spacing profile having a width wherein the sum of the tab spacing profile widths and the widths of all of the tabs in the at least two tabs equals the width of the device.

64. The device of claim 63 wherein the tab spacing profile between each of the tabs in the at least two tabs is the same.

65. The device of claim 63 having at least two different tab spacing profiles.

66. The device of claim 61 wherein when the device is disposed about a blood vessel in a body at least one of the two or more tabs of the first end is coupled to at least one of the two or more tabs on the second end.

67. The device of claim 63, wherein the tab widths and tab spacing profiles are selected such that when the at least one tab on the first end is coupled to at least one tab on the second end the device encircles a portion of a blood vessel and a side branch coupled to the blood vessel without restricting blood flow into or from the side branch.

68. The device of claim 67, wherein the portion of a blood vessel and a side branch comprises the descending aorta and at least one set of intercostal arteries.

69. The device of claim 67, wherein the portion of a blood vessel and a side branch comprises the inferior vena cava and at least one set of intercostal veins.

70. The device of claim 54, wherein the first end and the second end include cooperating portions of a mating fastener.

71. The device of claim 54, wherein the first end and the second end are configured to be sewn together.

72. The device of claim 70, wherein the mating fasteners are magnets.

73. The device of claim 70, wherein at least one of the mating fasteners is magnetic.

74. The device of claim 70, wherein a one the mating fasteners is a magnet and the other mating fastener is formed from a magnetically attractive material.

75. The device of claim 70, wherein the mating fasteners are opposite sides of a buckle.

76. The device of claim 70, wherein the mating fasteners are a screw and a screw-receiving opening.

77. The device of claim 70 wherein the mating fasteners are a hook and a loop.

78. The device of claim 70 wherein the mating fasteners comprise a plurality of hooks and a plurality of loops.

79. The device of claim 70 wherein the mating fasteners include a locking ring and a mating element.

80. The device of claim 70 wherein the mating fasteners include a positive-locks.

81. The device of claim 40, further comprising:
a conduit coupled to the second layer in communication with the opening, the conduit configured to be coupled to a pump.

82. The device of claim 81 wherein one of a fluid is configured to be selectively communicated in synchronization with the cardiac cycle to the cavity via a conduit in communication with the opening in the cover layer.

83. A vascular assist device configured to be coupled to at least a portion of a blood vessel, the device comprising:
a vascular engaging layer;
an expandable layer;
a cover layer; and
the device having an uninstalled configuration and an installed configuration;
wherein,
the vascular engaging layer is disposed between the outer wall of the blood vessel and the expanding layer;
the cover layer and the expanding layer are coupled so as to form a cavity therebetween, the cavity being bounded by the expanding layer and the cover layer;
the cover layer having an opening formed therein, the opening being in communication with the cavity and the cavity being configured to selectively receive a fluid via the opening whereby the fluid causes the volume of the cavity to change wherein the change in cavity volume causes the expanding layer to deform more than the cover layer to accommodate the change in cavity volume.

84. A vascular assist device according to claim 83 wherein the vascular engaging layer is sufficiently long to encircle a portion of the blood vessel.

85. A vascular assist device according to claim 83 wherein the expandable layer is sufficiently long to at least partially encircle a portion of the blood vessel.

86. A vascular assist device according to claim 83 wherein the vascular engaging layer is coupled to the expandable layer.

87. The device of claim 83, wherein the vascular engaging layer is fabricated with a first material, the expanding layer is fabricated with second material and the cover layer is fabricated with a third material different from the first and second material.

88. The device of claim 83, wherein the vascular engaging layer is a vascular graft.

89. The device of claim 88, wherein the vascular graft is made from a polymer selected from the group consisting of: polyester, nylon, polytetrafluoroethylene and polyvinylidene fluoride.

90. The device of claim 83, wherein the second material is a first silicone elastomer and the third material is a second silicone elastomer.

91. The device of claim 83, wherein the cover layer further comprises a reinforcement element selected from the group consisting of: polyester, nylon, para-aramid fiber, stainless steel, platinum, superelastic nitinol and alloys of nickel and titanium.

92. The device of claim 83 wherein the expanding layer and the cover layer are fabricated from a material selected from the group consisting of: silicone, neoprene, copolymers comprising styrene and copolymers comprising butadiene.

93. The device of claim 83, wherein the perimeter of the expanding layer is coupled to the cover layer.

94. The device of claim 93, wherein the perimeter of the expanding layer aligns with a portion of the perimeter of the cover layer.

95. The device of claim 83, wherein the device is repeatably configurable between the uninstalled configuration and the installed configuration.

96. The device of claim 95 wherein the cover layer further comprises at least one pair of cooperative fastening elements.

97. The device of claim 96 wherein when the device is in the uninstalled configuration the at least one pair of cooperative fastening elements are uncoupled.

98. The device of claim 96 wherein when the device is in the installed configuration the at least one pair of cooperative fastening elements are coupled.

99. The device of claim 96 wherein one of the fastening elements in the at least one pair of cooperative fastening elements comprises a plurality of fastening positions such that the size of the device in the installed configuration may be adjusted by changing to which of said plurality of fastening positions the other fastening element is coupled.

100. The device of claim 83 wherein the cover layer includes a first end and a second end, said first end and said second end configured to be removeably coupled such that the device is reconfigurable between an uninstalled configuration in which the first and second ends are separate and an installed configuration in which the first and second ends are coupled.

101. The device of claim 83 wherein the device is maintained in an installed configuration about a portion of a blood vessel by suturing a first portion of the vascular engaging layer to a second portion of the vascular engaging layer.

102. The device according to claim 83 wherein a portion of the device is coated with a tissue growth inducing polymeric material.

103. The device according to claim 102 wherein the tissue growth inducing polymeric material is one of poly-L-lysine and poly-D-lysine.

104. A vascular assist device configured to be coupled to at least a portion of a blood vessel, the device comprising:
a vascular engaging layer having a first stiffness;
a cover layer having a second stiffness that is greater than the first stiffness and being coupled to the vascular engaging layer such that a portion of the cover layer extends past at least a portion of the perimeter of the vascular engaging layer; the cover layer and the vascular engaging layer forming a cavity therebetween, the cover layer having an opening formed therein in communication with the cavity, the cavity being configured to selectively receive a fluid via the opening; and
the device having an uninstalled configuration and an installed configuration.

105. A vascular assist device according to claim 104 wherein the vascular engaging layer is sufficiently long to at least partially encircle a portion of the aorta.

106. A vascular assist device according to claim 104 wherein the vascular engaging layer is sufficiently long to at least partially encircle a portion of the vena cava.

107. The device of claim 104, wherein the vascular engaging layer is fabricated with a first material and the cover layer is fabricated with a second material different from the first material.

108. The device of claim 107, wherein the first material is a first silicone elastomer and the second material is a second silicone elastomer.

109. The device of claim 104, wherein the cover layer further comprises a reinforcement element selected from the group consisting of: polyester, nylon, para-aramid fiber, stainless steel, platinum, superelastic nitinol and alloys of nickel and titanium.

110. The device of claim 107, wherein the first and second material are one of silicone, neoprene, copolymers comprising styrene and copolymers comprising butadiene silicone, neoprene and copolymers comprising styrene and butadiene.

111. The device of claim 104, wherein the perimeter of the vascular engaging layer is coupled to the cover layer.

112. The device of claim 111, wherein the perimeter of the vascular engaging layer aligns with a portion of the perimeter of the cover layer.

113. The device of claim 111, wherein the perimeter of the cover layer extends beyond the perimeter of the vascular layer.

114. The device of claim 104, wherein the device is repeatably configurable between the uninstalled configuration and the installed configuration.

115. The device of claim 114 wherein the cover layer further comprises at least one pair of cooperative fastening elements.

116. The device of claim 115 wherein when the device is in the uninstalled configuration the at least one pair of cooperative fastening elements are uncoupled.

117. The device of claim 115 wherein when the device is in the installed configuration the at least one pair of cooperative fastening elements are coupled.

118. The device of claim 115 wherein one of the fastening elements in the at least one pair of cooperative fastening elements comprises a plurality of fastening positions such that the size of the device in the installed configuration may be adjusted by changing to which of said plurality of fastening positions the other fastening element is coupled.

119. The device of claim 104 wherein the cover layer includes a first end and a second end, said first end and said second end configured to be removeably coupled such that the device is reconfigurable between an uninstalled configuration in which the first and second ends are separate and an installed configuration in which the first and second ends are coupled.

120. The device according to claim 104 wherein a portion of the device is coated with a tissue growth inducing polymeric material.

121. The device according to claim 120 wherein the tissue growth inducing polymeric material is one of poly-L-lysine and poly-D-lysine.

122. A system, comprising:
a pump having a controller configured to receive a signal associated with the cardiac cycle of a heart;
a cuff comprising,
a compliant first layer configured to engage internal vasculature;
a second layer coupled to the first layer and having a stiffness greater than a stiffness of the first layer and having an opening formed therein;
the compliant first layer and the second layer being coupled to form a cavity bounded by the first layer and the second layer, the cavity being in communication with the opening in the second layer; and
a conduit coupled between the opening and the pump, the conduit configured to convey a fluid between the pump and the cavity thereby causing deformation of the first layer by expanding and contracting the cavity.

123. The system of claim 122 wherein the signal associated with the cardiac cycle is related to systole.

124. The system of claim 122 wherein the signal associated with the cardiac cycle is related to diastole.

125. The system of claim 122 wherein the signal associated with the cardiac cycle is related to a change in aortic pressure.

126. The system of claim 122 wherein the signal associated with the cardiac cycle is related to a change in arterial pressure.

127. The system of claim 122 wherein the signal associated with the cardiac cycle is related to a change in venous pressure.

128. The system of claim 122 wherein the pump is a pulsitile pump.

129. The system of claim 122 wherein the pump, cuff and conduit are implantable within a human body.

130. The system of claim 122 further comprising a sensor configured to generate a signal associated with the cardiac cycle of a heart.

131. The system of claim 130 herein the sensor is a pressure sensor.

132. The system of claim 131 wherein the sensor is part of the cuff.

133. The system of claim 130 wherein the sensor is an electrical sensor.

134. The system of claim 133 wherein the sensor is part of the cuff.

135. The system of claim 122 wherein the second layer includes a first end and a second end, said first end and said second end configured to be removeably coupled such that the cuff is reconfigurable between an uninstalled configuration in which the first and second ends are separate and an installed configuration in which the first and second ends are coupled.

136. The system of claim 135 wherein the installed configuration comprises a plurality of coupling positions whereby the size of the cuff may be adjusted by changing into which one of the plurality of coupling positions the first and second ends are coupled.

137. The system of claim 122, wherein the first layer is fabricated with a first material and the second layer is fabricated with a second material different from the first material.

138. The device of claim 122 wherein the second layer further comprising a first end and a second end, wherein each of the first end and the second end have at least two tabs, each of the tabs in the at least two tabs has a width wherein the sum of the widths of all the tabs in the at least two tabs on the first end is less than the width of the cuff.

139. The device of claim 138, wherein the at least two tabs on the first and second ends are configured to be removably coupled such that the device is reconfigurable between a first configuration in which the at least two tabs on the first and second ends are separate and a second configuration in which the at least two tabs on the first and second ends are coupled.

140. The device of claim 138 wherein a tab spacing profile is provided between adjacent tabs in the at least two tabs, the tab spacing profile having a width wherein the sum of the tab spacing profile widths and the widths of all of the tabs in the at least two tabs equals the width of the device.

141. The device of claim 140 wherein the tab spacing profile between each of the tabs in the at least two tabs is the same.

142. The device of claim 140 having at least two different tab spacing profiles.

143. The device of claim 138 wherein when the device is disposed about a blood vessel in a body at least one of the two or more tabs of the first end is coupled to at least one of the two or more tabs on the second end.

144. The device of claim 140, wherein the tab widths and tab spacing profiles are selected such that when the at least one tab on the first end is coupled to at least one tab on the second end the device encircles a portion of a blood vessel and a side branch coupled to the blood vessel without restricting blood flow into or from the side branch.

145. The device of claim 144, wherein the portion of a blood vessel and a side branch comprises the descending aorta and at least one set of intercostal arteries.

146. The device of claim 144, wherein the portion of a blood vessel and a side branch comprises the inferior vena cava and at least one set of intercostal veins.

147. The system of claim 122, wherein the conduit further comprises a first end configured to have a single lumen and a second end configured to have a plurality of lumens.

148. The system of claim 147, wherein each lumen has the same diameter.

149. The system of claim 147, wherein at least one lumen has a diameter different from the diameter of another lumen.

150. The system of claim 122, wherein the cavity is coupled to a plurality of conduits.

151. The system of claim 150, wherein one of the plurality of conduits supplies fluid from the pump to the cavity.

152. The system of claim 151, wherein another one of the plurality of conduits returns fluid from the cavity to the pump.

153. The system of claim 122, wherein the conduit has a first diameter adjacent to the opening and a second different diameter at a point distal to the opening.

154. The system of claim 122, wherein the conduit comprises a plurality of conduits wherein at least one of the conduits in the plurality of conduits has a diameter that is different from the diameter of at least one other of the plurlity of conduits.

155. The system of claim 137, wherein the first material is a first silicone elastomer and the second material is a second silicone elastomer.

156. The system of claim 122 wherein the fluid is a liquid is selected from the group consisting of: saline, water, glycols, a combination comprising a glycol and saline and a combination comprising a glycol and water.

157. The system of claim 122 wherein the fluid is a gas that is chemically inert with the first and second layers.

158. The system of claim 157 wherein the fluid is a gas that is one of either carbon dioxide or nitrogen.

159. The system of claim 122 wherein the fluid is a gas having lower density than air.

160. The system of claim 159 wherein the gas is helium.

161. The system of claim 122 further comprising a fluid volume compensator.

162. The system of claim 161 wherein the fluid conpensator is disposed in a fluid flow path between the pump and the cavity.

163. The system of claim 161 wherein the fluid compensator is configured to adjust the fluid volume ported into the cavity during activation of the cuff.

164. The system of claim 161 wherein the fluid compensator is configured to allow replenishment of the fluid in the system.

165. The system of claim 122 wherein a surface of one of the cuff, conduit and pump in contact with the fluid are coated with a material to enhance lubricity.

166. The system of claim 122 wherein a surface of one of the cuff, conduit and pump in contact with the fluid is coated with a material to reduce fluid evaporation.

167. The system of claim 122 wherein an interior surface of one of the cuff, conduit and pump is coated with a material to reduce fluid loss.

168. The system of claim 122 wherein a surface of one of the cuff, conduit and pump is coated with a material to reduce fluid loss.

169. The system of claim 122 wherein the condiut further comprises a reinforcement element.

170. The system of claim 169 wherein the reinforcement element is selected from the group consisting of: polyester, nylon, para-aramid fiber, stainless steel, platinum, superelastic nitinol and alloys of nickel and titanium.

171. A method for augmenting blood flow in a patient body using a cuff formed from a first layer joined to a second layer so that a cavity exists between the layers such that filling the cavity preferentially deforms the first layer, the method comprising:
detecting a first cardiac cycle trigger;
porting a fluid into the cavity so as to elastically deform the first layer thereby compressing a blood vessel in response to the first cardiac cycle trigger; and
porting a fluid out of the cavity in response to a second cardiac cycle trigger.

172. A method for augmenting blood flow in a body according to claim 171 wherein the first cardiac trigger is related to an ECG of the patient.

173. A method for augmenting blood flow in a body according to claim 171 wherein the first cardiac trigger is related to the increasing portion of the R-wave.

174. A method for augmenting blood flow in a body according to claim 173 wherein the first cardiac trigger occurs at 90% of the increasing R-wave amplitude.

175. A method for augmenting blood flow in a body according to claim 171 wherein the first cardiac trigger is related to the ECG of the patient and selected so that the step of porting a fluid into the cavity so as to elastically deform the first layer coincides with the ventricular systole.

176. A method for augmenting blood flow in a body according to claim 171 wherein the second cardiac cycle trigger is a predetermined time limit.

177. A method for augmenting blood flow in a body according to claim 171 wherein the second cardiac cycle trigger is based on the R-R interval.

178. A method for augmenting blood flow in a body according to claim 171 wherein the second cardiac cycle trigger is related to aortic pressure.

179. A method for augmenting blood flow in a body according to claim 171 wherein the first and the second cardiac cycle triggers are selected to operate the cuff in copulsation mode.

180. A method for augmenting blood flow in a body according to claim 171 wherein the cavity inflates during the ventricular systole of the heart.

181. A method for augmenting blood flow in a body according to claim 171 wherein the first cardiac trigger is related to the Q-T interval.

182. A method for augmenting blood flow in a body according to claim 171 wherein the first cardiac trigger is related to the decreasing portion of the T-wave.

183. A method for augmenting blood flow in a body according to claim 182 wherein the first cardiac trigger occurs at the end of the T-wave.

184. A method for augmenting blood flow in a body according to claim 171 wherein the first cardiac trigger is related to the T-wave and selected so that the step of porting a fluid into the cavity so as to elastically deform the first layer coincides with the ventricular diastole.

185. A method for augmenting blood flow in a body according to claim 181 wherein the second cardiac cycle trigger is a predetermined time limit.

186. A method for augmenting blood flow in a body according to claim 181 wherein the second cardiac cycle trigger is based on the R-R interval.

187. A method for augmenting blood flow in a body according to claim 181 wherein the second cardiac cycle trigger is related to aortic pressure.

188. A method for augmenting blood flow in a body according to claim 171 wherein the first and the second cardiac cycle triggers are selected to operate the cuff in counterpulsation mode.

189. A method for augmenting blood flow in a body according to claim 171 wherein the cavity inflates during the ventricular diastole of the heart.

190. A method for augmenting blood flow in a body using a cuff formed from a first layer joined to a second layer so that a cavity exists between the layers such that filling the cavity preferentially deforms the first layer, the method comprising:
detecting a cardiac cycle trigger;
porting a fluid into the cavity so as to elastically deform the first layer thereby compressing a blood vessel in response to the cardiac cycle trigger; holding the vessel compressed for known duration and
porting a fluid out of the cavity at the end of the duration in order to allow the vessel to relax.

191. A method for augmenting blood flow in a body according to claim 190 wherein the cardiac trigger is related to the ECG.

192. A method for augmenting blood flow in a body according to claim 190 wherein the cardiac trigger is related to the increasing portion of the R-wave of the ECG.

193. A method for augmenting blood flow in a body according to claim 192 in a coplusation manner wherein the first cardiac trigger occurs at 90% of the increasing R-wave amplitude.

194. A method for augmenting blood flow in a body according to claim 190 in a coplusation manner wherein the cardiac trigger is related to the aortic pressure and selected so that the step of porting a fluid into the cavity so as to elastically deform the first layer coincides with the ventricular systole.

195. A method for augmenting blood flow in a body according to claim 190 in counterpulsation manner, wherein the cardiac trigger is related to detecting R-wave of the ECG, computing the Q-T interval and triggering the pump to coincide with the end of the T-wave for porting the fluid into the cavity so as to elastically deform the first layer and compress the blood vessel.

196. A method for augmenting blood flow in a body according to claim 190 in counterpulsation manner, wherein the cardiac trigger is related to detecting the peak aortic pressure and computing the duration for the aortic valve to close and triggering the pump for porting the fluid into the cavity so as to elastically deform the first layer and compress the blood vessel to coincide with the aortic valve closing.

197. A method for augmenting blood flow in a vessel of a patient using a cuff having a compliant first layer that at least partially encircles a vessel adjacent the cuff, a second layer coupled to the first layer, the first layer and the second layer defining a cavity therebetween, the method comprising:
changing the pressure of a fluid in the cavity based on a signal associated with the cardiac cycle;
deforming the first layer in response to the changing pressure of the fluid in the cavity; and
deforming the walls of a vessel at least partially encircled by the first layer in response to the deforming of the first layer.

198. A method for augmenting blood flow in a vessel according to claim 197 wherein the signal associated with the cardiac cycle is related to the ECG of the patient.

199. A method for augmenting blood flow in a vessel according to claim 197 wherein the signal associated with the cardiac cycle is related to the increasing portion of the R-wave.

200. A method for augmenting blood flow in a vessel according to claim 197 wherein the signal associated with the cardiac cycle is related to the ECG of the patient and selected so that the step of deforming the walls of a vessel at least partially encircled by the first layer in response to the deforming of the first layer coincides with the ventricular systole.

201. A method for augmenting blood flow in a vessel according to claim 197 wherein the signal associated with the cardiac cycle is related to aortic pressure.

202. A method for augmenting blood flow in a vessel according to claim 197 wherein the changing the pressure of a fluid in the cavity is selected such that the blood flow in the vessel is augmented in a copulsation mode.

203. A method for augmenting blood flow in a vessel according to claim 197 wherein the changing the pressure of a fluid in the cavity is occurring so that the pressure in the cavity is increasing during the ventricular systole of the heart.

204. A method for augmenting blood flow in a vessel according to claim 197 wherein the signal associated with the cardiac cycle is related to the Q-T interval.

205. A method for augmenting blood flow in a vessel according to claim 197 wherein the signal associated with the cardiac cycle is related to the decreasing portion of the T-wave.

206. A method for augmenting blood flow in a vessel according to claim 197 wherein the signal associated with the cardiac cycle occurs at the end of the T-wave.

207. A method for augmenting blood flow in a vessel according to claim 197 wherein the signal associated with the cardiac cycle is related to the T-wave and selected so that the step of changing the pressure of a fluid in the cavity coincides with the ventricular diastole.

208. A method for augmenting blood flow in a vessel according to claim 197 wherein the changing the pressure of a fluid in the cavity is selected such that the blood flow in the vessel is augmented in a counterpulsation mode.

209. A method for augmenting blood flow in a vessel according to claim 197 wherein the changing the pressure of a fluid in the cavity is occurring so that the pressure in the cavity is increasing during the ventricular diastole of the heart.

210. A method for augmenting blood flow in a vessel according to claim 197 wherein increasing the pressure in the cavity results in deforming the first layer so as to constrict the vessel.

211. A system, comprising:
a plurality of cuffs, each of the plurality of cuffs including
a compliant first layer configured to engage internal vasculature;
a second layer coupled to the first layer, the first layer and the second layer defining a cavity therebetween, the second layer defining an opening in communication with the cavity; and
a connector configured to couple the plurality of cuffs to one another.

212. The system of claim 211, wherein the connector is coupled to the second layer of each of the plurality of cuffs.

213. The system of claim 211, wherein the connector further comprises a conduit coupled between the connector and an opening.

214. The system of claim 211, wherein at least one of the plurality of cuffs is coupled to the vasculature of a body.

215. The system of claim 214, wherein at least one of the plurality of cuffs is coupled to an organ in a body.

216. The system of claim 211 wherein at least one of the plurality of cuffs is configured to engage with at least one set of intercostal arteries.

217. The system of claim 211 wherein at least one of the plurality of cuffs is configured to engage with at least one set of intercostal veins.

218. The system of claim 211 wherein at least one of the plurality of cuffs is configured to engage with the ascending aorta.

219. The system of claim 211 wherein at least one of the plurality of cuffs is configured to engage with the descending aorta.

220. The system of claim 211, wherein plurality of cuffs are configured to engage with the superior vena cava.

221. The system of claim 211, wherein plurality of cuffs are configured to engage with the inferior vena cava.

222. The system of claim 211, wherein each of the plurality of cuffs is repeatably configurable between an uninstalled configuration and an installed configuration.

223. The system of claim 222 wherein the second layer of each of the plurality of cuffs further comprises at least one pair of cooperative fastening elements.

224. The system of claim 223 wherein when a cuff is in the uninstalled configuration the at least one pair of cooperative fastening elements are uncoupled.

225 The system of claim 223 wherein when a cuff is in the installed configuration the at least one pair of cooperative fastening elements are coupled.

226. The system of claim 223 wherein one of the fastening elements in the at least one pair of cooperative fastening elements comprises a plurality of fastening positions such that the size of the cuff in the installed configuration may be adjusted by changing to which of said plurality of fastening positions the other fastening element is coupled.

227. The system of claim 211 wherein the second layer of each of the plurality of cuffs includes a first end and a second end, said first end and said second end configured to be removeably coupled such that the cuff is reconfigurable between an uninstalled configuration in which the first and second ends are separate and an installed configuration in which the first and second ends are coupled.

228. The system of claim 211 further comprising:
a pump in communication with the connector; and
a controller for providing control signals to the pump in response to triggering signals from a cardiac cycle.

229. The system of claim 228 configured so that each of the plurality of cuffs is operated sequentially.

230. The system of claim 228 configured so that each of the plurality of cuffs is operated simultaneously.

231. The system of claim 228 configured so that each of the plurality of cuffs is operated to augment blood flow in the internal vasculature in a counterpulsation mode.

232. The system of claim 228 configured so that each of the plurality of cuffs is operated to augment blood flow in the internal vasculature in a copulsation mode.
